(19) United States
(12) Patent Application Publication
(10) Pub. No.: US 2022/0135981 A1
(43) Pub. Date: May 5, 2022

(54) TARGETING SENESCENT CELLS

(71) Applicant: INSTITUT DE CARDIOLOGIE DE MONTREAL, Montréal (CA)

(72) Inventor: Éric THORIN, Montréal (CA)

(21) Appl. No.: 17/433,284

(22) PCT Filed: Feb. 27, 2020
(86) PCT No.: PCT/CA2020/050261

§ 371 Date: Aug. 24, 2021

Related U.S. Application Data
(60) Provisional application No. 62/811,057, filed on Feb. 27, 2019.

(51) Int. Cl. C12N 15/113; A61K 45/06; A61P 9/10; A61K 31/713

(52) U.S. Cl. CPC C12N 15/1136; A61K 45/06; A61P 9/10; C12N 2320/30; C12N 2310/531; C12N 2310/14; A61K 31/713

(57) ABSTRACT

Described herein are methods and compositions for treating a subject having a disease associated with an angiopoietin like-2 (angptl2) positive (angptl2+) senescent cell by administering an agent that induces death of the angptl2+ senescent cell.

### BACKGROUND OF THE INVENTION

During aging, mortality rate rises exponentially because of a loss of normal organ functions, including tissue maintenance and repair capacity. Stressors that young tissues may tolerate because of their functional reserve become insurmountable in the elderly, leading to age-related diseases. Age-related diseases are of clinical concern as the global population ages: 38% of people between 40 to 60 years old, and 83% of those over 85 years have cardiovascular diseases. This will worsen with 1 billion individuals projected to be over 65 years old by 2030. Vascular endothelial dysfunction is one of the first asymptomatic defect occurring with aging, a change that is measurable in humans by the age of 45 to 50 years old in the general population, and earlier in men than women. Endothelial dysfunction is characterized by a reduction in 1) flow-mediated dilation essential to match organ metabolic needs, 2) anti-aggregation and -adhesion of platelets and circulating immune cells, essential to delay atherogenesis, and 3) its barrier function, that regulates exchanges, hormonal regulations and immune responses. Thus, any chronic alteration of the endothelium will have a deleterious impact on organ function because the endothelium interacts directly or indirectly with all other cell types to meet their needs and responds to the environment.

At the cellular level, aging of healthy vascular endothelial cells (EC)s leads to senescence, a state of permanent growth arrest. Senescence of cells, such as ECs, is found to be directly related to the risk for several diseases. There is unmet need in the field to develop methods for treating such senescent cell-associated diseases by targeting senescent cells, while leaving healthy cells and healthy tissues unharmed.

### SUMMARY OF THE INVENTION

Featured herein are methods for treating senescent cell (SnC)-associated diseases by using a SnC-targeting agent that induces cell death (e.g., apoptosis) of one or more SnC. Particularly featured are methods for treating SnC-associated diseases by using an angiopoietin like-2 (angptl2) positive (angptl2⁺) SnC-targeting agent that induces cell death (e.g., apoptosis) of one or more angptl2⁺ SnC.

A first aspect features a method of treating a disease associated with an angptl2⁺ senescent cell (e.g., a hepatocyte, a cardiac cell, a glial cell, a neuron, a synovial cell, or an endothelial cell (EC)) in a subject (e.g., a human) in need thereof by administering to the subject an agent that induces cell death (e.g., apoptosis) of the angptl2⁺ senescent cell. In some embodiments of the first aspect, the agent is a vector (e.g., a viral vector, such as an adeno-associated virus (AAV)) encoding a small hairpin RNA (shRNA).

In some embodiments of the first aspect, the cell is a cardiac cell; the agent is an AAV serotype 1 (AAV1) or AAV serotype 9 (AAV9) encoding a shRNA; the disease is a cardiovascular disease (e.g., atherosclerosis); and/or the method further includes administering a second therapeutic agent (e.g., an antihypertensive agent or a cholesterol lowering agent). In some embodiments, the featured method reduces atherosclerotic lesions and/or reduces atherogenesis in the subject.

In some embodiments of the first aspect, the cell is a hepatocyte; the agent is an AAV serotype 8 (AAV8) encoding a shRNA; and/or the disease is a hepatic disease (e.g., hepatic steatosis, such as non-alcoholic steato-hepatosis (NASH)). In some embodiments, the featured method reduces liver triglyceride level in the subject.

In some embodiments of the first aspect, the cell is a brain cell and/or the disease is a cerebrovascular disease (e.g., vascular cognitive impairment and dementia (VCID)).

In some embodiments of the first aspect, the disease is an autoimmune disease (e.g., arthritis or psoriasis).

In some embodiments of the first aspect, the method: reduces expression (e.g., mRNA expression or protein expression) of angptl2; increases endothelial repair; reduces senescence-associated secretory phenotype (SASP, such as expression of Pai-1); reduces inflammation; and/or increases lifespan of the subject.

Another aspect features a pharmaceutical composition including an agent that induces cell death (e.g., apoptosis) of an angptl2⁺ senescent cell for use in treating a disease associated with angptl2⁺ senescent cells in a subject (e.g., a human) in need thereof. In some embodiments of this aspect, the agent is a vector (e.g., a viral vector, such as an AAV) encoding a shRNA.

In some embodiments of the above aspect, the agent is an AAV1 or AAV9 encoding a shRNA; the disease is a cardiovascular disease (e.g., atherosclerosis); and/or the pharmaceutical composition further includes a second therapeutic agent (e.g., an antihypertensive agent or a cholesterol lowering agent). In some embodiments, the featured pharmaceutical composition reduces atherosclerotic lesions and/or reduces atherogenesis in the subject.

In some embodiments of the above aspect, the agent is an AAV8 encoding a shRNA and/or the disease is a hepatic disease (e.g., hepatic steatosis, such as NASH). In some embodiments, the featured pharmaceutical composition reduces liver triglyceride level in the subject.

In some embodiments of the above aspect, the disease is a cerebrovascular disease (e.g., VCID). In some embodiments, the disease is an autoimmune disease (e.g., arthritis or psoriasis).

In some embodiments of the above aspect, the pharmaceutical composition further includes a therapeutically acceptable carrier.

In some embodiments of the above aspect, the pharmaceutical composition: reduces expression (e.g., mRNA expression or protein expression) of angptl2; increases endothelial repair; reduces SASP (e.g., Pai-1 expression); reduces inflammation; and/or increases lifespan of the subject.

In some embodiments of any of the above the aforementioned aspects, the subject is a human.

### Definitions

As described herein “senescence” refers to a cellular phenomenon wherein a proliferative-competent cell (e.g., an endothelial cell) undergoes permanent growth arrest in response to various cellular stresses. Cellular senescence is typically associated with aging, but can also occur prematurely following exposure to multiple types of stress, such as oxidative stress, DNA damage, mitogenic stress and metabolic stress. Cells undergoing senescence, cells with senescence, and/or cells exhibiting one or more functional, phenotypic, and/or molecular signatures of senescence (e.g., expression (e.g., mRNA and/or protein expression) of one or more molecular mediators and/or markers of senescence, such as p21, p53, and p16) are collectively referred to herein as “senescent cells,” “SnC” or “SnCs.” Examples of SnC include senescent endothelial cells (SnECs), senescent brain cells, senescent hepatocytes, senescent cardiac cells, senescent glial cells, senescent neurons, and senescent synovial cells.

As used herein, “senescent endothelial cell” or “SnEC” refers to endothelial cell (EC) undergoing senescence, EC with senescence, and/or EC exhibiting one or more functional, phenotypic, and/or molecular signatures of senescence (e.g., expression (e.g., mRNA and/or protein expression) of one or more molecular mediators and/or markers of senescence, such as p21, p53, and p16).

As used herein, “senescence-associated secretory phenotype” or “SASP” refers to factors, released by SnC that contribute to one or more outcomes of senescence (e.g., chronic inflammation, organ dysfunction, or organ failure). For example, Pai-1 is a prominent SASP factor.

As used herein, “SnC-associated disease” refers to a disease caused by cellular and/or organ dysfunction stemming from senescence. SnC-associated diseases are characterized by one or more functional, phenotypic, and/or molecular signatures of senescence. Examples of SnC-associated diseases include cardiovascular disease (e.g., atherosclerosis), hepatic disease (e.g., hepatic steatosis, such as non-alcoholic steato-hepatosis (NASH)), cerebrovascular disease (e.g., vascular cognitive impairment and dementia (VCID)) and autoimmune disease (e.g., arthritis or psoriasis). A SnC-associated disease is treated by one or more methods described herein

As used herein, an “angptl2⁺ SnC” or “angptl2⁺ SnEC” refer to a SnC or a SnEC that expresses angiopoietin-like-2 (angptl2), a member of the angiopoietin-like (angptl) family. An “angptl2⁺ SnC” or “angptl2⁺ SnEC may express angptl2 at mRNA and/or protein level. An angptl2+ SnC or an angptl2⁺ SnEC is targeted for treatment of a SnC-associated disease in one or more methods described herein.

As used herein, the term “agent” refers to nucleic acid (e.g., RNA or DNA), protein, antibody, vector construct, viral vector, cell or small molecule that is used in the methods and compositions described herein for a specific purpose (e.g., to achieve a particular outcome). SnC-targeting agent (e.g., angptl2⁺ SnC-targeting agent) is an example of an agent that is used for the purpose of targeting a SnC (e.g., an angptl2⁺ SnC) by the methods and compositions described herein, For example, a shRNA (e.g., shAngptl2) is used as an agent (e.g., a SnC-targeting agent, such as an angptl2⁺ SnC-targeting agent) for the purpose of targeting a SnC (e.g., an angptl2⁺ SnC) by the methods and compositions described herein,

As used herein, the term “cell death” or “death” refers to programmed cell death or death of one or more cells mediated in a regulated manner by an intracellular program. Examples of cell death include apoptosis or type I cell-death, autophagy or type II cell-death, and necroptosis. For example, the methods and composition described herein leads to cell death (e.g., apoptosis) of SnC (e.g., angptl2⁺ SnC, such as angptl2⁺ SnEC).

As used herein, the terms “increase” or “increasing” and “decrease” or “decreasing” refer to modulating resulting in, respectively, greater or lesser amounts, of function, expression level, or activity of a metric relative to a reference. For example, subsequent to administration of one or more agents in the methods described herein, one or more cellular response (e.g., response of endothelial cell, such as apoptosis, endothelial repair, or expression of SASP) may increase or decrease in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to those metrics prior to administration of the agent. Generally, the metric is measured subsequent to administration at a time that the administration has had the recited effect, e.g., at least one week, one month, 3 months, or 6 months, after a treatment regimen has begun. The term “reducing” is used interchangeably with the term “decreasing” herein.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, inhalation routes, and direct absorption through mucous membrane tissues. The therapeutic agents is administered by the same route or by different routes. For example, a first therapeutic agent of the combination may be administered by intravenous injection while a second therapeutic agent of the combination may be administered orally.

As used herein, the terms “effective amount,” “therapeutically effective amount,” and a “sufficient amount” of a composition or agent described herein refer to a quantity sufficient to, when administered to a subject, including a mammal (e.g., a human), effect beneficial or desired results, including effects at the cellular level, tissue level, or clinical results, and, as such, an “effective amount” or synonym thereto depends upon the context in which it is being applied. For example, in the context of treating a SnC-associated disease, it is an amount of the composition or agent sufficient to achieve a treatment response as compared to the response obtained without administration of the composition or agent. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition or an agent of the present disclosure is an amount that results in a beneficial or desired result in a subject as compared to a control. As defined herein, a therapeutically effective amount of a composition or an agent of the present disclosure may be readily determined by one of ordinary skill by routine methods known in the art. Dosage regimen may be adjusted to provide the optimum therapeutic response.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and which is indicated for human use.

As used herein, the term “reference” refers to a level, expression level, copy number, sample or standard that is used for comparison purposes. For example, a reference sample is obtained from a healthy individual (e.g., an individual who does not have a SnC-associated disease). A reference level is the level of expression of one or more reference samples. For example, an average expression (e.g., a mean expression or median expression) among a plurality of individuals (e.g., healthy individuals). In other instances, a reference level is a predetermined threshold level, e.g., based on functional expression as otherwise determined, e.g., by empirical assays.

As used herein, the term “sample” refers to a specimen (e.g., blood, blood component (e.g., serum or plasma), urine, saliva, amniotic fluid, cerebrospinal fluid, tissue (e.g., barrier tissue, skin, gut tissue, airway tissue, wound tissue, placental, or dermal), pancreatic fluid, chorionic villus sample, and cells) isolated from a subject.

As used herein, the terms “subject” and “patient” refer to a mammal, such as a human. A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “about” refers to a value that is ±10% of the recited value.

Throughout this specification and claims, the word “comprise,” or variations such as “comprises” or “comprising,” will be understood to imply the inclusion of a stated integer or group of integers but not the exclusion of any other integer or group of integers.

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments, e.g., Fab, Fab′ and F(ab′)₂, Fd, Fvs, single-chain Fvs (scFv), rlgG, single-chain antibodies, disulfide-linked Fvs (sdFv), fragments including either a V_{L} or V_{H} domain, fragments produced by an Fab expression library, and anti-idiotypic (anti-Id) antibodies. Antibody molecules of the invention is of any type (e.g., IgG, IgE, IgM, IgD, IgA, and IgY), class (e.g., IgG1, IgG2, IgG3, IgG4, IgA1 and IgA2) or subclass of immunoglobulin molecule. Moreover, unless otherwise indicated, the term “monoclonal antibody” (mAb) is meant to include both intact molecules as well as antibody fragments (such as, for example, Fab and F (ab′)₂ fragments) that are capable of specifically binding to a target antigen. Fab and F (ab′)₂ fragments lack the Fc fragment of an intact antibody.

Other features and advantages of the invention will be apparent from the following Detailed Description and the claims.

### BRIEF DESCRIPTION OF THE DRAWINGS

FIG. 1 depicts the immunofluorescence staining for mCherry in frozen aortic sections of ATX mice at 6 months of age, 3 months after the mice were injected with adeno-associated virus serotype 1 (AAV1)-shAngptl2 or AAV1-shScramble (shSCR). The level of mCherry is shown in red, basal lamina is shown in green, and nuclei are shown in blue.

FIG. 2 is a graph showing mRNA expression of cardiac and liver Angptl2 in shAngptl2 ATX mice, 3 months after the mice were injected with AAV1-shAngptl2 or AAV1-shSCR. Average gene expression level in shSCR mice was arbitrarily set at 1. Data is mean±SEM of n ATX mice.

FIG. 3 is a series of graphs showing cholesterol, triglyceride, and glucose levels of ATX mice at the age of 6 months, 3 months after the mice were injected with AAV1-shAngptl2 or AAV1-shSCR. Data is mean±SEM of n=7 ATX mice.

FIG. 4 is a graphical representation of the quantification of thoracic aorta atheroma plaque area, and representative images of longitudinally open thoracic aortas of 3 month-old (-mo) control (CTL 3-mo, n=6), 6-mo control (CTL 6-mo, n=12), 6-mo AAV1-shScramble (+shSCR, n=11) and 6-mo AAV1-shAngptl2 (+shAngptl2, n=12) ATX mice revealing white atheroma plaques at 6-mo. Data is mean±SEM. *: p<0.0001 vs. CTL 6-mo and shSCR; ^{S}: p<0.0001 vs. CTL 3-mo; 1-way ANOVA, Tukey's multiple comparison test.

FIG. 5A and FIG. 5B are graphs showing mRNA expression of p21, Pai-1 and angptl2 in the native aortic endothelium of ATX mice. FIG. 5A is a graphical representation of plaque growth (area) in the aorta, and mRNA expression of p21, Pai-1 and angptl2 in the native aortic endothelium of 3-mo (n=4), 5-mo (n=4), 6-mo (n=4) and 12-mo (n=4) ATX mice. The average level of gene expression in 3-mo ATX mice was arbitrarily set at 1. Plaque area was quantified from longitudinally open thoracic aortas of 3-mo (n=7), 5-mo (n=5), 6-mo (n=7), 9-mo (n=12) and 12-mo (n=4) ATX mice. Data are expressed as mean±SEM. *: p<0.0001 vs. 3-mo ATX mice. FIG. 5B is a graphical representation of mRNA expression of p21, Pai-1 and angptl2 in the native aortic endothelium Of 6-mo WT and ATX mice (n=3). The average level of gene expression in 6-mo WT mice was arbitrarily set at 1. Data are expressed as mean±SEM. *: p<0.05 vs. WT mice; *** P<0.001 vs WT (t test).

FIG. 6 is a graph depicting mRNA expression of Angptl2, p21, Pai-1, Cd68, Icam-1, 11-1 #and Mcp1 in the native aortic endothelium isolated from AAV1-shAngptl2-treated ATX mice. Data is presented as means with Min to Max. ‡: p=0.10; *: p<0.05; **: p=0.019; ***: p=0.0095 vs. shSCR group (unpaired Mann-Whitney test, two-tailed).

FIG. 7 is a graph representing mRNA expression of Angptl2, p21, Pai-1, Cd68, 11-1 #and Mcp1 in the de-endothelialized aortic wall isolated from shAngptl2-treated ATX mice.

Data is mean±SEM. ‡: p=0.057; *: p<0.03 and **: p=0.017 vs. shSCR group (unpaired Mann-Whitney test, two-tailed).

FIG. 8A, FIG. 8B, and FIG. 8C are graphs depicting mRNA expression of Angptl2 (FIG. 8A), p21 (FIG. 8B) and Pai-1 (FIG. 8C) post-injections in the native endothelium freshly harvested from thoracic aortas of ATX mice treated or not with AAV1-shSCR or AAV1-shAngptl2. Average gene expression level in shSCR group was arbitrarily set at 1. Data is mean±SEM. *: p<0.05; **: p<0.01;***: p<0.001 vs. shSCR group.

FIG. 9A and FIG. 9B are graphs depicting mRNA expression of Bax (FIG. 9A, p=0.005) and Bcl2 (FIG. 9B, p=0.0027) of Angptl2⁺ SnEC in aortas of shAngptl2-treated ATX mice, 1-week post-injection. Data is mean±SEM (unpaired t test, two-tailed).

FIG. 10A is a graphical representation of mRNA expression of the progenitor marker Cd34 in the native aortic endothelium of shAngptl2 ATX mice, 4-week post-injection. Data is mean±SEM. *: p=0.0342 vs. shSCR group (unpaired t test, two-tailed). The average gene expression level in the shSCR group was arbitrarily set at 1.

FIG. 10B depicts the immunofluorescence staining for CD34 in the endothelium of 6-mo shAngptl2-treated or shSCR-treated ATX mice 3 months post injection, and graphical representation thereof. Data are mean±SEM. *: p<0.024 vs. shSCR.

FIG. 11 is a survival curve showing health span in male Angptl2 knockdown (KD, n=26) mice compared to their male wild type (WT, n=20) littermates (p=0.0401, Gehan-Breslow-Wilcoxon test).

FIG. 12A, FIG. 12B, FIG. 12C, and FIG. 12D are graphical representations of linear correlations between ANGPTL2 and p21 mRNA expression (FIG. 12A), age of the donors and ANGPTL2 and p21 mRNA expression (FIG. 12B), ANGPTL2 and TNF-α, IL-8 and IL-6 mRNA expression (FIG. 12C), and p21 and TNF-α, IL-8 and IL-6 mRNA expression (FIG. 12D) in IMA segments (n=26) from atherosclerotic patients undergoing coronary artery by-pass surgery. Non-parametric Spearman correlation test were applied. Patient characteristics are described in Table 3, herein.

### DETAILED DESCRIPTION

Cellular senescence refers to the specific phenomenon wherein a proliferative-competent cell (e.g., an endothelial cell) undergoes permanent growth arrest in response to various cellular stresses. Cellular senescence is a stress-responsive, adaptive phenotype that develops through multiple stages, including major metabolic and secretory readjustments, which can spread from cell to cell and can occur at any point during life. Although typically associated with aging, cellular senescence can also occur prematurely following exposure to multiple types of stress, such as oxidative stress, DNA damage, mitogenic stress and metabolic stress. Senescence is characterized by specific changes in cell morphology and gene expression, which leads to cellular dysfunction. Despite cell growth arrest, senescent cells (SnC) remain metabolically active and release numerous potentially harmful factors referred to collectively as the senescence-associated secretory phenotype (SASP). SASP factors, released by accumulated SnC, likely contribute to chronic inflammation, induce senescence of neighboring cells and contribute to organ dysfunction or failure. Cellular and/or organ dysfunction stemming from senescence has been linked to various diseases, collectively referred to herein as senescent cell (SnC)-associated diseases. Disclosed herein are methods of treating a SnC-associated disease by specific targeting of a SnC.

### Senescent Cells

Senescence of cells (e.g., endothelial cells (EC), hepatocytes, cardiac cells, brain cells, glial cells, neurons, and synovial cells) is triggered by high oxidative stress, DNA damage and telomerase inactivation, and directly related to the burden of risks for various senescent cell (SnC)-associated diseases. In one or more methods described herein, the SnC that is targeted for treatment of a SnC-associated disease may be a senescent endothelial cell (SnEC), a senescent hepatocyte, a senescent cardiac cell, a senescent brain cell, a senescent glial cell, a senescent neuron, or a senescent synovial cell. A member of the angiopoietin-like (angptl) family, angiopoietin-like-2 (angptl2), has been identified as a biomarker of SnC, such as SnEC (Thorin et al., U.S. Pat. No. 7,972,795; incorporated herein by reference in its entirety). Angptl2 is shown to play a major pro-inflammatory role in a variety of pathologies, including atherosclerosis, diabetes, abdominal aortic aneurysm, neointimal hyperplasia, rheumatoid arthritis, dermatomyositis, tumor progression and endothelial dysfunction. Increased angptl2 expression has been reported in endothelial cells from chronic atherosclerotic smokers, while its circulating level correlates with adiposity, C-reactive protein levels, and tumor necrosis factor (TNF)α levels (Yu et al., J Am Heart Assoc, 3: e001024, 2014; incorporated by reference in its entirety). In one or more methods described herein, the SnC that is targeted for treatment of a SnC-associated disease may be an angptl2⁺ SnC. Specifically, the SnC that is targeted for treatment of a SnC-associated disease in one or more methods described herein is an angptl2⁺ SnEC, an angptl2⁺ senescent hepatocyte, an angptl2⁺ senescent cardiac cell, an angptl2+ senescent brain cell, an angptl2⁺ senescent glial cell, an angptl2⁺ senescent neuron, or an angptl2+ senescent synovial cell. In particular, the SnC that is targeted for treatment of a SnC-associated disease in one or more methods described herein is an angptl2⁺ SnEC.

### SnC-Associated Diseases

The SnC-associated disease that is treated by the methods and compositions described herein is an angptl2⁺ SnC-associated disease. The SnC-associated disease that is treated by the methods and compositions described herein can be a SnEC-associated disease. In particular, the SnC-associated disease (e.g., SnEC-associated disease) that is treated by the methods and compositions described herein is an angptl2⁺ SnC-associated disease (e.g., angptl2⁺ SnEC-associated disease). The SnC-associated disease (e.g., angptl2⁺ SnC-associated disease) that is treated by the methods and compositions described herein can be a cardiovascular disease (CVD) (e.g., atherosclerosis), a hepatic disease (e.g., hepatic steatosis, such as non-alcoholic steato-hepatosis (NASH)), a cerebrovascular disease (e.g., vascular cognitive impairment and dementia (VCID)), or an autoimmune disease (e.g., arthritis or psoriasis).

Specifically, the SnC-associated disease (e.g., angptl2⁺ SnC-associated disease) that is treated by the methods and compositions described herein may be a CVD. In particular, a CVD (e.g., angptl2⁺ SnC-associated CVD) that is treated by the methods and compositions described herein may be atherosclerosis (e.g., atherosclerosis in a subject, such as a human). Atherosclerosis, such as angptl2⁺ SnC-associated atherosclerosis (e.g., angptl2⁺ senescent cardiac cell-associated atherosclerosis and/or angptl2⁺ SnEC-associated atherosclerosis) that is treated by the methods and compositions described herein may exhibit (e.g., in a subject, such as a human) one or more of the following indications: (i) increased expression and/or release of SASP factors, such as Pai-1, angptl2, and inflammatory cytokines or chemokines (e.g., IL-13, IL-12, MCP-1, ICAM-1, TNF-α, IL-8, and/or IL-6); (ii) increased inflammation (e.g., chronic inflammation); (iii) increased senescence (e.g., increase in p21, p53 and/or p16 expression (e.g., mRNA and/or protein expression)); (iv) increased accumulation of SnC (e.g., angptl2⁺ SnC, such as angptl2⁺ senescent cardiac cells and/or angptl2+ SnEC), such as accumulation due to reduced clearance (e.g., due to reduced immune surveillance) and/or increase in senescence; (v) reduced endothelial function; (vi) reduced endothelial repair; (vii) reduced cardiac repair; (viii) increased atherogenesis; (ix) increased growth of atheroma plaque; (x) increased atherosclerotic burden (e.g., in thoracic aorta); (xi) increased incidence of and/or risk of developing atrial fibrillation; (xii) increased incidence of and/or risk of heart failure; (xiii) increased incidence of and/or risk of developing VCID; and/or (xiv) reduced healthspan and/or lifespan.

In certain embodiments, one or more of the indications exhibited by atherosclerosis is increased or decreased in a subject (e.g., a human with atherosclerosis) by 5% or more (e.g., 5%, 10%, 15%, 20%, 25%, 30%, 40%, 50%, 60%, 70%, 80%, 90%, 95%, 99%, or more), compared to a healthy control (e.g., a healthy human). In certain embodiments, the indication is increased or decreased in a subject (e.g., a human with atherosclerosis) between 5-20%, between 5-50%, between 10-50%, between 10-80%, between 20-80%, or between 20-100%.

Alternatively, the SnC-associated disease (e.g., angptl2⁺ SnC-associated disease) that is treated by the methods and compositions described herein may be a hepatic disease. In particular, a hepatic disease (e.g., angptl2⁺ SnC-associated hepatic disease) that is treated by the methods and compositions described herein may be hepatic steatosis (e.g., hepatic steatosis in a subject, such as a human). Specifically, hepatic steatosis that is treated by the methods and compositions described herein may be NASH (e.g., NASH in a subject, such as a human). Alternatively, hepatic steatosis that is treated by the methods and compositions described herein may be alcoholic fatty liver disease (e.g., alcoholic fatty liver disease in a subject, such as a human). Hepatic steatosis, such as angptl2+ SnC-associated hepatic steatosis (e.g., angptl2⁺ senescent hepatocyte-associated hepatic steatosis and/or angptl2⁺ SnEC-associated hepatic steatosis) that is treated by the methods and compositions described herein may exhibit one or more of the following (e.g., in a subject, such as a human): (i) increased expression (e.g., mRNA and/or protein expression) and/or release of SASP factors, such as angptl2, and inflammatory cytokines or chemokines (e.g., IL-13, IL-12, MCP-1, ICAM-1, TNF-α, IL-8, and/or IL-6); (ii) increased inflammation (e.g., chronic inflammation); (iii) increased senescence (e.g., increase in p21, p53 and/or p16 expression (e.g., mRNA and/or protein expression)); (iv) increased accumulation of SnC (e.g., angptl2⁺ SnC, such as angptl2⁺ senescent hepatocytes and/or angptl2+ SnEC), such as accumulation due to reduced clearance (e.g., due to reduced immune surveillance) and/or increase in senescence; (v) reduced endothelial function; (vi) reduced endothelial repair; (vii) reduced hepatocyte repair; (viii) increased liver triglyceride level; (ix) increased size of adipocytes; and/or (x) reduced healthspan and/or lifespan.

In certain embodiments, one or more of the indications exhibited by hepatic steatosis is increased or decreased in a subject (e.g., a human with hepatic steatosis) by 5% or more (e.g., 5%, 10%, 15%, 20%, 25%, 30%, 40%, 50%, 60%, 70%, 80%, 90%, 95%, 99%, or more), compared to a healthy control (e.g., a healthy human). In certain embodiments, the indication is increased or decreased in a subject (e.g., a human with hepatic steatosis) between 5-20%, between 5-50%, between 10-50%, between 10-80%, between 20-80%, or between 20-100%.

Alternatively, the SnC-associated disease (e.g., angptl2⁺ SnC-associated disease) that is treated by the methods and compositions described herein may be a cerebrovascular disease. In particular, a cerebrovascular disease (e.g., angptl2⁺ SnC-associated cerebrovascular disease) that is treated by the methods and compositions described herein may be VCID (e.g., VCID in a subject, such as a human). VCID that is treated by the methods and compositions described herein may be angptl2⁺ SnC-associated VCID (e.g., angptl2⁺ senescent brain cell-associated VCID and/or angptl2+ SnEC-associated VCID).

In certain embodiments, one or more indications exhibited by VCID is increased or decreased in a subject (e.g., a human with VCID) by 5% or more (e.g., 5%, 10%, 15%, 20%, 25%, 30%, 40%, 50%, 60%, 70%, 80%, 90%, 95%, 99%, or more), compared to a healthy control (e.g., a healthy human). In certain embodiments, the indication is increased or decreased in a subject (e.g., a human with VCID) between 5-20%, between 5-50%, between 10-50%, between 10-80%, between 20-80%, or between 20-100%.

Alternatively, the SnC-associated disease (e.g., angptl2⁺ SnC-associated disease) that is treated by the methods and compositions described herein may be an autoimmune disease. In particular, an autoimmune disease (e.g., angptl2⁺ SnC-associated autoimmune disease) that is treated by the methods and compositions described herein may be arthritis (e.g., arthritis in a subject, such as a human) or psoriasis (e.g., psoriasis in a subject, such as a human). An autoimmune disease (e.g., arthritis or psoriasis) that is treated by the methods and compositions described herein may be an angptl2⁺ SnC-associated autoimmune disease (e.g., angptl2⁺ SnEC-associated autoimmune disease).

In certain embodiments, one or more indications exhibited by an autoimmune disease (e.g., arthritis or psoriasis) is increased or decreased in a subject (e.g., a human with an autoimmune disease) by 5% or more (e.g., 5%, 10%, 15%, 20%, 25%, 30%, 40%, 50%, 60%, 70%, 80%, 90%, 95%, 99%, or more), compared to a healthy control (e.g., a healthy human). In certain embodiments, the indication is increased or decreased in a subject (e.g., a human with an autoimmune disease) between 5-20%, between 5-50%, between 10-50%, between 10-80%, between 20-80%, or between 20-100%.

### Treatment of Angptl2⁺ SnC-Associated Diseases

Featured herein are methods and compositions for treating one or more SnC-associated diseases (e.g., angptl2⁺ SnC-associated diseases) by targeting angptl2⁺ SnC (e.g., angptl2⁺ SnEC, angptl2⁺ senescent hepatocytes, angptl2⁺ senescent cardiac cells, angptl2⁺ senescent brain cells, angptl2⁺ senescent glial cells, angptl2⁺ senescent neurons, or angptl2⁺ senescent synovial cells). One of the key anticipated benefits of the featured methods is the reduction of adverse side effects during treatment (e.g., treatment of atherosclerosis or hepatic steatosis) achieved by specific targeting of the SnC (e.g., angptl2⁺ SnC), while leaving healthy cells and healthy tissues unharmed.

Targeting angptl2⁺ SnC

In the methods and compositions described herein, a SnC (e.g., an angptl2⁺ SnC) is targeted with one or more SnC-targeting agents (e.g., an angptl2⁺ SnC-targeting agent) for the treatment of a SnC-associated disease (e.g., an angptl2⁺ SnC-associated disease).

In some embodiments, a SnC-targeting agent (e.g., an angptl2⁺ SnC-targeting agent) described herein can target a SnC (e.g., an angptl2⁺ SnC) and induce cell death (e.g., apoptosis, autophagy, and/or necroptosis) of the SnC. Specifically, an angptl2⁺ SnC-targeting agent described herein can induce cell death (e.g., apoptosis, autophagy, and/or necroptosis) of an angptl2⁺ SnC (e.g., an angptl2⁺ SnEC, an angptl2⁺ senescent hepatocyte, an angptl2⁺ senescent cardiac cell, an angptl2+ senescent brain cell, an angptl2⁺ senescent glial cell, an angptl2⁺ senescent neuron, or an angptl2+ senescent synovial cell) in one or more methods described herein. For example, an angptl2⁺ SnC-targeting agent (e.g., an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) described herein can induce cell death (e.g., apoptosis, autophagy, and/or necroptosis) of an angptl2⁺ SnC in one or more methods described herein. In some embodiments, a SnC-targeting agent (e.g., an angptl2⁺ SnC-targeting agent, such as an angptl2⁺ SnEC-targeting agent, an angptl2⁺ senescent hepatocyte-targeting agent, an angptl2+ senescent cardiac cell-targeting agent, an angptl2⁺ senescent brain cell-targeting agent, an angptl2+ senescent glial cell-targeting agent, an angptl2⁺ senescent neuron-targeting agent, or an angptl2+ senescent synovial cell-targeting agent) described herein can induce or increase cell death (e.g., apoptosis, autophagy, and/or necroptosis) of SnC (e.g., angptl2⁺ SnC, such as angptl2⁺ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2⁺ senescent synovial cell) by at least 5% or more (e.g., 5%, 10%, 15%, 20%, 25%, 30%, 40%, 50%, 60%, 70%, 80%, 90%, 95%, 99%, or more), compared to control SnC that are not targeted by the SnC-targeting agent. In certain embodiments, cell death (e.g., apoptosis, autophagy, and/or necroptosis) of SnC (e.g., angptl2⁺ SnC, such as angptl2⁺ SnEC, angptl2⁺ senescent hepatocyte, angptl2⁺ senescent cardiac cell, angptl2+ senescent brain cell, angptl2⁺ senescent glial cell, angptl2⁺ senescent neuron, or angptl2⁺ senescent synovial cell) that are targeted by a SnC-targeting agent (e.g., an angptl2⁺ SnC-targeting agent, such as an angptl2⁺ SnEC-targeting agent, an angptl2⁺ senescent hepatocyte-targeting agent, an angptl2+ senescent cardiac cell-targeting agent, an angptl2⁺ senescent brain cell-targeting agent, an angptl2+ senescent glial cell-targeting agent, an angptl2⁺ senescent neuron-targeting agent, or an angptl2+ senescent synovial cell-targeting agent) is increased between 5-20%, between 5-50%, between 10-50%, between 20-80%, between 20-100%, between 50-200%, or between 100%-500%.

In particular, a SnC-targeting agent (e.g., an angptl2⁺ SnC-targeting agent, such as an angptl2⁺ SnEC-targeting agent, an angptl2⁺ senescent hepatocyte-targeting agent, an angptl2+ senescent cardiac cell-targeting agent, an angptl2⁺ senescent brain cell-targeting agent, an angptl2+ senescent glial cell-targeting agent, an angptl2⁺ senescent neuron-targeting agent, or an angptl2+ senescent synovial cell-targeting agent) described herein can induce or increase apoptosis of an SnC (e.g., angptl2⁺ SnC, such as angptl2⁺ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2⁺ senescent synovial cell) in one or more methods described herein. In some embodiments, a SnC-targeting agent (e.g., an angptl2⁺ SnC-targeting agent, such as an angptl2⁺ SnEC-targeting agent) described herein can induce or increase apoptosis of SnCs (e.g., angptl2⁺ SnC, such as angptl2⁺ SnEC) by at least 5% or more (e.g., 5%, 10%, 15%, 20%, 25%, 30%, 40%, 50%, 60%, 70%, 80%, 90%, 95%, 99%, or more), compared to control SnCs that are not targeted by the SnC-targeting agent. In certain embodiments, apoptosis of SnC (e.g., angptl2⁺ SnC, such as angptl2⁺ SnEC) that are targeted by a SnC-targeting agent (e.g., an angptl2⁺ SnC-targeting agent, such as an angptl2+ SnEC-targeting agent) is increased between 5-20%, between 5-50%, between 10-50%, between 20-80%, between 20-100%, between 50-200%, or between 100%-500%. In some embodiments, a SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) described herein can increase the expression (e.g., mRNA expression or protein expression) of a pro-apoptotic gene (e.g., Bax). For example, administration of a SnC-targeting agent (e.g., an angptl2+ SnC-targeting agent, such as an angptl2⁺ SnEC-targeting agent) to a subject (e.g., a human) can increase the expression (e.g., mRNA expression or protein expression) of a pro-apoptotic gene (e.g., Bax) by 5% or more (e.g., 5%, 10%, 15%, 20%, 25%, 30%, 40%, 50%, 60%, 70%, 80%, 90%, 95%, 99%, or more), compared to the expression in the subject before administration of the SnC-targeting agent, or a subject (e.g., a human) to which the SnC-targeting agent is not administered. In some embodiments, a SnC-targeting agent (e.g., an angptl2⁺ SnC-targeting agent, such as an angptl2+ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) described herein can reduce the expression (e.g., mRNA expression or protein expression) of an anti-apoptotic gene (e.g., Bcl2). For example, administration of a SnC-targeting agent (e.g., an angptl2⁺ SnC-targeting agent, such as an angptl2⁺ SnEC-targeting agent) to a subject (e.g., a human) can reduce the expression (e.g., mRNA expression or protein expression) of an anti-apoptotic gene (e.g., Bcl2) by 5% or more (e.g., 5%, 10%, 15%, 20%, 25%, 30%, 40%, 50%, 60%, 70%, 80%, 90%, 95%, 99%, or more), compared to the expression in the subject before administration of the SnC-targeting agent, or a subject (e.g., a human) to which the SnC-targeting agent is not administered.

Alternatively, a SnC-targeting agent (e.g., an angptl2⁺ SnC-targeting agent, such as an angptl2⁺ SnEC-targeting agent, an angptl2⁺ senescent hepatocyte-targeting agent, an angptl2+ senescent cardiac cell-targeting agent, an angptl2⁺ senescent brain cell-targeting agent, an angptl2+ senescent glial cell-targeting agent, an angptl2⁺ senescent neuron-targeting agent, or an angptl2+ senescent synovial cell-targeting agent) described herein can induce or increase autophagy of an SnC (e.g., angptl2⁺ SnC, such as angptl2⁺ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2⁺ senescent synovial cell) in one or more methods described herein. In some embodiments, a SnC-targeting agent (e.g., an angptl2⁺ SnC-targeting agent, such as an angptl2⁺ SnEC-targeting agent) described herein can induce or increase autophagy of SnCs (e.g., angptl2⁺ SnC, such as angptl2⁺ SnEC) by at least 5% or more (e.g., 5%, 10%, 15%, 20%, 25%, 30%, 40%, 50%, 60%, 70%, 80%, 90%, 95%, 99%, or more), compared to control SnCs that are not targeted by the SnC-targeting agent. In certain embodiments, autophagy of SnCs (e.g., angptl2⁺ SnC, such as angptl2⁺ SnEC) that are targeted by a SnC-targeting agent (e.g., an angptl2⁺ SnC-targeting agent, such as an angptl2+ SnEC-targeting agent) is increased between 5-20%, between 5-50%, between 10-50%, between 20-80%, between 20-100%, between 50-200%, or between 100%-500%.

In other embodiments, a SnC-targeting agent (e.g., an angptl2⁺ SnC-targeting agent, such as an angptl2⁺ SnEC-targeting agent, an angptl2⁺ senescent hepatocyte-targeting agent, an angptl2+ senescent cardiac cell-targeting agent, an angptl2⁺ senescent brain cell-targeting agent, an angptl2+ senescent glial cell-targeting agent, an angptl2⁺ senescent neuron-targeting agent, or an angptl2+ senescent synovial cell-targeting agent) described herein can induce or increase necroptosis of an SnC (e.g., angptl2⁺ SnC, such as angptl2⁺ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2⁺ senescent synovial cell) in one or more methods described herein. In some embodiments, a SnC-targeting agent (e.g., an angptl2⁺ SnC-targeting agent, such as an angptl2⁺ SnEC-targeting agent) described herein can induce or increase necroptosis of SnCs (e.g., angptl2⁺ SnC, such as angptl2⁺ SnEC) by at least 5% or more (e.g., 5%, 10%, 15%, 20%, 25%, 30%, 40%, 50%, 60%, 70%, 80%, 90%, 95%, 99%, or more), compared to control SnCs that are not targeted by the SnC-targeting agent. In certain embodiments, necroptosis of SnCs (e.g., angptl2⁺ SnC, such as angptl2⁺ SnEC) that are targeted by a SnC-targeting agent (e.g., an angptl2⁺ SnC-targeting agent, such as an angptl2+ SnEC-targeting agent) is increased between 5-20%, between 5-50%, between 10-50%, between 20-80%, between 20-100%, between 50-200%, or between 100%-500%.

In some embodiments, a SnC-targeting agent (e.g., an angptl2⁺ SnC-targeting agent) described herein can reduce or inhibit the function and/or activity of a SnC (e.g., an angptl2⁺ SnC). Specifically, an angptl2⁺ SnC-targeting agent (e.g., an angptl2⁺ SnEC-targeting agent, an angptl2+ senescent hepatocyte-targeting agent, an angptl2⁺ senescent cardiac cell-targeting agent, an angptl2+ senescent brain cell-targeting agent, an angptl2⁺ senescent glial cell-targeting agent, an angptl2+ senescent neuron-targeting agent, or an angptl2⁺ senescent synovial cell-targeting agent) described herein can reduce or inhibit the function and/or activity of an angptl2⁺ SnC (e.g., angptl2⁺ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2⁺ senescent synovial cell) in one or more methods described herein. In some embodiments, a SnC-targeting agent (e.g., an angptl2+ SnC-targeting agent, such as an angptl2⁺ SnEC-targeting agent) described herein can reduce or inhibit the function and/or activity of SnCs (e.g., angptl2⁺ SnC, such as angptl2⁺ SnEC) by 5% or more (e.g., 5%, 10%, 15%, 20%, 25%, 30%, 40%, 50%, 60%, 70%, 80%, 90%, 95%, 99%, or more), compared to control SnCs that are not targeted by the SnC-targeting agent. In certain embodiments, function and/or activity of SnCs (e.g., angptl2⁺ SnC, such as angptl2⁺ SnEC) that are targeted by a SnC-targeting agent (e.g., an angptl2⁺ SnC-targeting agent, such as an angptl2⁺ SnEC-targeting agent) is reduced or inhibited between 5-20%, between 5-50%, between 10-50%, between 10-80%, between 20-80%, or between 20-100%.

In some embodiments, a SnC-targeting agent (e.g., an angptl2⁺ SnC-targeting agent) described herein can reduce or inhibit angptl2 expression (e.g., mRNA and/or protein expression) in a SnC (e.g., an angptl2⁺ SnC). Specifically, an angptl2⁺ SnC-targeting agent (e.g., an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) described herein can reduce or inhibit angptl2 expression (e.g., mRNA and/or protein expression) in an angptl2⁺ SnC (e.g., angptl2⁺ SnEC, angptl2⁺ senescent hepatocyte, angptl2+ senescent cardiac cell, angptl2⁺ senescent brain cell, angptl2⁺ senescent glial cell, angptl2⁺ senescent neuron, or angptl2⁺ senescent synovial cell) in one or more methods described herein. In some embodiments, a SnC-targeting agent (e.g., an angptl2⁺ SnC-targeting agent, such as an angptl2+ SnEC-targeting agent) described herein can reduce or inhibit angptl2 expression (e.g., mRNA and/or protein expression) in SnCs (e.g., angptl2⁺ SnC, such as angptl2⁺ SnEC) by 5% or more (e.g., 5%, 10%, 15%, 20%, 25%, 30%, 40%, 50%, 60%, 70%, 80%, 90%, 95%, 99%, or more), compared to control SnCs that are not targeted by the SnC-targeting agent. In certain embodiments, angptl2 expression (e.g., mRNA and/or protein expression) in SnCs (e.g., angptl2⁺ SnC, such as angptl2+ SnEC) that are targeted by a SnC-targeting agent (e.g., an angptl2⁺ SnC-targeting agent, such as an angptl2⁺ SnEC-targeting agent) is reduced or inhibited between 5-20%, between 5-50%, between 10-50%, between 10-80%, between 20-80%, or between 20-100%.

In some embodiments, a SnC-targeting agent (e.g., an angptl2⁺ SnC-targeting agent) described herein can reduce or inhibit angptl2 signaling of a SnC (e.g., an angptl2⁺ SnC). Specifically, an angptl2⁺ SnC-targeting agent (e.g., an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) described herein can reduce or inhibit angptl2 signaling of an angptl2⁺ SnC (e.g., angptl2⁺ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2⁺ senescent synovial cell) in one or more methods described herein. In some embodiments, a SnC-targeting agent (e.g., an angptl2⁺ SnC-targeting agent, such as an angptl2⁺ SnEC-targeting agent) described herein can reduce or inhibit angptl2 signaling of SnCs (e.g., angptl2⁺ SnC, such as angptl2⁺ SnEC) by 5% or more (e.g., 5%, 10%, 15%, 20%, 25%, 30%, 40%, 50%, 60%, 70%, 80%, 90%, 95%, 99%, or more), compared to control SnCs that are not targeted by the SnC-targeting agent. In certain embodiments, angptl2 signaling of SnCs (e.g., angptl2⁺ SnC, such as angptl2⁺ SnEC) that are targeted by a SnC-targeting agent (e.g., an angptl2⁺ SnC-targeting agent, such as an angptl2⁺ SnEC-targeting agent) is reduced or inhibited between 5-20%, between 5-50%, between 10-50%, between 10-80%, between 20-80%, or between 20-100%.

Treating angptl2⁺ SnC-Associated Disease

The methods and compositions described herein is used to target a SnC (e.g., an angptl2+ SnC) for treating a SnC-associated disease (e.g., an angptl2⁺ SnC-associated disease) in a subject by administering to the subject an effective amount of a SnC-targeting agent (e.g., an angptl2⁺ SnC-targeting agent) described herein. The method may include administering locally or systemically to the subject a SnC-targeting agent (e.g., an angptl2⁺ SnC-targeting agent) described herein in a dose (e.g., effective amount) and for a time sufficient to treat the SnC-associated disease (e.g., an angptl2+ SnC-associated disease).

The SnC-targeting agents (e.g., angptl2⁺ SnC-targeting agent, such as shAngptl2) can treat a SnC-associated disease (e.g., an angptl2⁺ SnC-associated disease) by inducing or increasing cell death (e.g., apoptosis, autophagy, and/or necroptosis) of one or more SnCs (e.g., angptl2⁺ SnCs) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase cell death (e.g., apoptosis, autophagy, and/or necroptosis) of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can induce or increase cell death (e.g., apoptosis, autophagy, and/or necroptosis) of SnCs (e.g., angptl2⁺ SnCs) in a subject or cell culture between 5-20%, between 5-50%, between 10-50%, between 20-80%, between 20-100%, between 50-200%, or between 100%-500%.

In particular, the SnC-targeting agents (e.g., angptl2⁺ SnC-targeting agent, such as shAngptl2) can treat a SnC-associated disease (e.g., an angptl2⁺ SnC-associated disease) by inducing or increasing apoptosis of one or more SnCs (e.g., angptl2⁺ SnCs) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase apoptosis of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can induce or increase apoptosis of SnCs (e.g., angptl2⁺ SnCs) in a subject or cell culture between 5-20%, between 5-50%, between 10-50%, between 20-80%, between 20-100%, between 50-200%, or between 100%-500%.

In some embodiments, the SnC-targeting agents (e.g., angptl2⁺ SnC-targeting agent, such as shAngptl2) can treat a SnC-associated disease (e.g., an angptl2⁺ SnC-associated disease) by inducing or increasing autophagy of one or more SnCs (e.g., angptl2⁺ SnCs) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase autophagy of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can induce or increase autophagy of SnCs (e.g., angptl2⁺ SnCs) in a subject or cell culture between 5-20%, between 5-50%, between 10-50%, between 20-80%, between 20-100%, between 50-200%, or between 100%-500%.

In some embodiments, the SnC-targeting agents (e.g., angptl2⁺ SnC-targeting agent, such as shAngptl2) can treat a SnC-associated disease (e.g., an angptl2⁺ SnC-associated disease) by inducing or increasing necroptosis of one or more SnCs (e.g., angptl2⁺ SnCs) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase necroptosis of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can induce or increase necroptosis of SnCs (e.g., angptl2⁺ SnCs) in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat a SnC-associated disease (e.g., an angptl2⁺ SnC-associated disease) by reducing expression (e.g., mRNA and/or protein expression) of angptl2 (e.g., expression of angptl2 in a SnC, such as an angptl2⁺ SnC) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce expression (e.g., mRNA and/or protein expression) of angptl2 (e.g., expression of angptl2 in a SnC, such as an angptl2⁺ SnC) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce expression (e.g., mRNA and/or protein expression) of angptl2 (e.g., expression of angptl2 in a SnC, such as an angptl2⁺ SnC) in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat a SnC-associated disease (e.g., an angptl2⁺ SnC-associated disease) by reducing expression (e.g., mRNA and/or protein expression) and/or release of one or more SASP factors, such as Pai-1, inflammatory cytokines or chemokines (e.g., angptl2, IL-13, IL-12, MCP-1, ICAM-1, TNF-α, IL-8, and/or IL-6) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce expression (e.g., mRNA and/or protein expression) and/or release of one or more SASP factors by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce expression (e.g., mRNA and/or protein expression) and/or release of one or more SASP factors in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat a SnC-associated disease (e.g., an angptl2⁺ SnC-associated disease) by reducing inflammation (e.g., reducing expression (e.g., mRNA and/or protein expression) of one or more inflammatory mediators) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce inflammation by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce inflammation in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat a SnC-associated disease (e.g., an angptl2⁺ SnC-associated disease) by reducing senescence (e.g., reducing expression (e.g., mRNA and/or protein expression) of one or more senescence mediators and/or senescence markers (e.g., p21, p53, or p16)) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce senescence by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce senescence in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat a SnC-associated disease (e.g., an angptl2⁺ SnC-associated disease) by reducing accumulation of SnC (e.g., angptl2⁺ SnC, such as angptl2⁺ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2⁺ senescent synovial cell) or increasing clearance of SnC (e.g., angptl2⁺ SnC, such as angptl2+ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2⁺ senescent synovial cell) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce accumulation of SnC or increase clearance of SnC by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce accumulation of SnC in a subject or cell culture between 5-20%, between 5-50%, between 10-50%, between 10-80%, between 20-80%, or between 20-100%. A SnC-targeting agent (e.g., angptl2⁺ SnC-targeting agent) can increase clearance of SnC in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat a SnC-associated disease (e.g., an angptl2⁺ SnC-associated disease) by increasing immune surveillance (e.g., leading to increased clearance and/or reduced accumulation of SnC (e.g., angptl2+ SnC, such as angptl2⁺ SnEC, angptl2⁺ senescent hepatocyte, angptl2⁺ senescent cardiac cell, angptl2⁺ senescent brain cell, angptl2⁺ senescent glial cell, angptl2⁺ senescent neuron, or angptl2+ senescent synovial cell)) in a subject (e.g., a human subject or animal model) or in a cell culture (e.g., a culture generated from a patient sample, or a repository of patient samples). A SnC-targeting agent (e.g., angptl2⁺ SnC-targeting agent) can increase immune surveillance by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase immune surveillance in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat a SnC-associated disease (e.g., an angptl2⁺ SnC-associated disease) by increasing endothelial function in a subject (e.g., a human subject or animal model) or in a cell culture (e.g., a culture generated from a patient sample, or a repository of patient samples). A SnC-targeting agent (e.g., angptl2⁺ SnC-targeting agent) can increase endothelial function by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can increase endothelial function in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat a SnC-associated disease (e.g., an angptl2⁺ SnC-associated disease) by increasing endothelial repair in a subject (e.g., a human subject or animal model) or in a cell culture (e.g., a culture generated from a patient sample, or a repository of patient samples). A SnC-targeting agent (e.g., angptl2⁺ SnC-targeting agent) can increase endothelial repair by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase endothelial repair in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat a SnC-associated disease (e.g., an angptl2⁺ SnC-associated disease) by increasing healthspan and/or lifespan of a subject (e.g., a human subject or animal model) or a cell culture (e.g., a culture generated from a patient sample, or a repository of patient samples). A SnC-targeting agent (e.g., angptl2⁺ SnC-targeting agent) can increase healthspan and/or lifespan by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase healthspan and/or lifespan in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat a SnC-associated disease (e.g., an angptl2⁺ SnC-associated disease) by reducing one or more symptoms or indications of a SnC-associated disease (e.g., an angptl2⁺ SnC-associated disease) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce one or more symptoms or indications of a SnC-associated disease (e.g., an angptl2⁺ SnC-associated disease) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce one or more symptoms or indications of a SnC-associated disease (e.g., an angptl2+ SnC-associated disease) in a subject or cell culture between 5-20%, between 5-50%, between 10-50%, between 10-80%, between 20-80%, or between 20-100%.

In specific embodiments, the SnC-associated disease (e.g., angptl2⁺ SnC-associated disease) that is treated by targeting a SnC (e.g., angptl2⁺ SnC, such as angptl2⁺ senescent cardiac cell, angptl2⁺ senescent hepatocyte, angptl2⁺ senescent brain cell, and/or angptl2⁺ SnEC) with one or more SnC-targeting agents (e.g., an angptl2⁺ SnC-targeting agent, such as angptl2⁺ senescent cardiac cell-targeting agent, angptl2⁺ senescent hepatocyte-targeting agent, angptl2⁺ senescent brain cell-targeting agent, and/or angptl2⁺ SnEC-targeting agent) in the methods described herein is a CVD (e.g., atherosclerosis), a hepatic disease (e.g., hepatic steatosis, such as NASH), a cerebrovascular disease (e.g., VCID), or an autoimmune disease (e.g., arthritis or psoriasis). The methods and compositions described herein is used to target a SnC (e.g., an angptl2⁺ SnC) for treating a CVD (e.g., atherosclerosis), a hepatic disease (e.g., hepatic steatosis, such as NASH), a cerebrovascular disease (e.g., VCID), or an autoimmune disease (e.g., arthritis or psoriasis) in a subject by administering to the subject an effective amount of a SnC-targeting agent (e.g., an angptl2⁺ SnC-targeting agent) described herein. The method may include administering locally or systemically to the subject a SnC-targeting agent (e.g., an angptl2⁺ SnC-targeting agent) described herein in a dose (e.g., effective amount) and for a time sufficient to treat the CVD (e.g., atherosclerosis), or hepatic disease (e.g., hepatic steatosis, such as NASH), cerebrovascular disease (e.g., VCID), or autoimmune disease (e.g., arthritis or psoriasis).

(i) Treating Atherosclerosis

In specific embodiments, the methods and compositions described herein is used to target a SnC (e.g., an angptl2⁺ SnC, such as angptl2⁺ senescent cardiac cell and/or angptl2⁺ SnEC) for treating atherosclerosis in a subject (e.g., a human, such as a patient with atherosclerosis) by administering to the subject an effective amount of a SnC-targeting agent (e.g., an angptl2⁺ SnC-targeting agent, such as shAngptl2) described herein. The method may include administering locally or systemically to the subject a SnC-targeting agent (e.g., an angptl2⁺ SnC-targeting agent) described herein in a dose (e.g., effective amount) and for a time sufficient to treat the atherosclerosis.

The SnC-targeting agents (e.g., angptl2⁺ SnC-targeting agent, such as shAngptl2) can treat atherosclerosis by inducing or increasing cell death (e.g., apoptosis, autophagy, and/or necroptosis) of one or more SnCs (e.g., angptl2⁺ SnC, such as such as angptl2⁺ senescent cardiac cell and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase cell death (e.g., apoptosis, autophagy, and/or necroptosis) of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can induce or increase cell death (e.g., apoptosis, autophagy, and/or necroptosis) of SnCs (e.g., angptl2⁺ SnCs) in a subject or cell culture between 5-20%, between 5-50%, between 10-50%, between 20-80%, between 20-100%, between 50-200%, or between 100%-500%.

In particular, the SnC-targeting agents (e.g., angptl2⁺ SnC-targeting agent, such as shAngptl2) can treat atherosclerosis by inducing or increasing apoptosis of one or more SnCs (e.g., angptl2⁺ SnCs, such as angptl2⁺ senescent cardiac cell and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase apoptosis of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can induce or increase apoptosis of SnCs (e.g., angptl2⁺ SnCs) in a subject or cell culture between 5-20%, between 5-50%, between 10-50%, between 20-80%, between 20-100%, between 50-200%, or between 100%-500%.

In some embodiments, the SnC-targeting agents (e.g., angptl2⁺ SnC-targeting agent, such as shAngptl2) can treat atherosclerosis by inducing or increasing autophagy of one or more SnCs (e.g., angptl2⁺ SnCs, such as angptl2⁺ senescent cardiac cell and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase autophagy of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can induce or increase autophagy of SnCs (e.g., angptl2⁺ SnCs) in a subject or cell culture between 5-20%, between 5-50%, between 10-50%, between 20-80%, between 20-100%, between 50-200%, or between 100%-500%.

In some embodiments, the SnC-targeting agents (e.g., angptl2⁺ SnC-targeting agent, such as shAngptl2) can treat atherosclerosis by inducing or increasing necroptosis of one or more SnCs (e.g., angptl2⁺ SnCs, such as angptl2⁺ senescent cardiac cell and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase necroptosis of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can induce or increase necroptosis of SnCs (e.g., angptl2⁺ SnCs) in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat atherosclerosis by reducing expression (e.g., mRNA and/or protein expression) of angptl2 (e.g., expression of angptl2 in a SnC, such as an angptl2⁺ SnC (e.g., angptl2⁺ senescent cardiac cell and/or angptl2⁺ SnEC)) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce expression (e.g., mRNA and/or protein expression) of angptl2 (e.g., expression of angptl2 in a SnC, such as an angptl2⁺ SnC) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce expression (e.g., mRNA and/or protein expression) of angptl2 (e.g., expression of angptl2 in a SnC, such as an angptl2⁺ SnC) in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing expression (e.g., mRNA and/or protein expression) and/or release of one or more SASP factors, such as Pai-1, inflammatory cytokines or chemokines (e.g., angptl2, IL-13, IL-12, MCP-1, ICAM-1, TNF-α, IL-8, and/or IL-6) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce expression (e.g., mRNA and/or protein expression) and/or release of one or more SASP factors by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce expression (e.g., mRNA and/or protein expression) and/or release of one or more SASP factors in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing inflammation (e.g., reducing expression (e.g., mRNA and/or protein expression) of one or more inflammatory mediators) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce inflammation by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce inflammation in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing senescence (e.g., reducing expression (e.g., mRNA and/or protein expression) of one or more senescence mediators and/or senescence markers (e.g., p21, p53, or p16)) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce senescence by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce senescence in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing accumulation of SnC (e.g., angptl2⁺ SnC, such as angptl2⁺ senescent cardiac cell and/or angptl2⁺ SnEC) or increasing clearance of SnC (e.g., angptl2⁺ SnC, such as angptl2⁺ senescent cardiac cell and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce accumulation of SnC or increase clearance of SnC by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce accumulation of SnC in a subject or cell culture between 5-20%, between 5-50%, between 10-50%, between 10-80%, between 20-80%, or between 20-100%. A SnC-targeting agent (e.g., angptl2⁺ SnC-targeting agent) can increase clearance of SnC in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat atherosclerosis by increasing immune surveillance (e.g., leading to increased clearance and/or reduced accumulation of SnC (e.g., angptl2⁺ SnC, such as angptl2⁺ senescent cardiac cell and/or angptl2⁺ SnEC)) in a subject (e.g., a human subject or animal model) or in a cell culture (e.g., a culture generated from a patient sample, or a repository of patient samples). A SnC-targeting agent (e.g., angptl2⁺ SnC-targeting agent) can increase immune surveillance by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase immune surveillance in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat atherosclerosis by increasing endothelial function in a subject (e.g., a human subject or animal model) or in a cell culture (e.g., a culture generated from a patient sample, or a repository of patient samples). A SnC-targeting agent (e.g., angptl2⁺ SnC-targeting agent) can increase endothelial function by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase endothelial function in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat atherosclerosis by increasing endothelial repair in a subject (e.g., a human subject or animal model) or in a cell culture (e.g., a culture generated from a patient sample, or a repository of patient samples). A SnC-targeting agent (e.g., angptl2⁺ SnC-targeting agent) can increase endothelial repair by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase endothelial repair in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat atherosclerosis by increasing cardiac cell repair in a subject (e.g., a human subject or animal model) or in a cell culture (e.g., a culture generated from a patient sample, or a repository of patient samples). A SnC-targeting agent (e.g., angptl2⁺ SnC-targeting agent) can increase cardiac cell repair by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase cardiac cell repair in a subject or cell culture between 5-20%, between 5-50%, between 10-50%, between 20-80%, between 20-100%, between 50-200%, or between 100%-500%. The SnC-targeting agents (e.g., angptl2⁺ SnC-targeting agent, such as shAngptl2) can treat atherosclerosis by reducing atherogenesis in a subject (e.g., a human subject or animal model) or in a cell culture (e.g., a culture generated from a patient sample, or a repository of patient samples). A SnC-targeting agent (e.g., angptl2⁺ SnC-targeting agent) can reduce atherogenesis by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce atherogenesis in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing growth of atheroma plaque in a subject (e.g., a human subject or animal model) or in a cell culture (e.g., a culture generated from a patient sample, or a repository of patient samples). A SnC-targeting agent (e.g., angptl2⁺ SnC-targeting agent) can reduce growth of atheroma plaque by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce growth of atheroma plaque in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing atherosclerotic burden (e.g., in thoracic aorta) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce atherosclerotic burden by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce atherosclerotic burden in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing the incidence of and/or risk of developing atrial fibrillation in a subject (e.g., a human subject or animal model) or in a cell culture (e.g., a culture generated from a patient sample, or a repository of patient samples). A SnC-targeting agent (e.g., angptl2⁺ SnC-targeting agent) can reduce the incidence of and/or risk of developing atrial fibrillation by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce the incidence of and/or risk of developing atrial fibrillation in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing the incidence of and/or risk of heart failure in a subject (e.g., a human subject or animal model) or in a cell culture (e.g., a culture generated from a patient sample, or a repository of patient samples). A SnC-targeting agent (e.g., angptl2⁺ SnC-targeting agent) can reduce the incidence of and/or risk of heart failure by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce the incidence of and/or risk of heart failure in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by reducing the incidence of and/or risk of developing VCID in a subject (e.g., a human subject or animal model) or in a cell culture (e.g., a culture generated from a patient sample, or a repository of patient samples). A SnC-targeting agent (e.g., angptl2⁺ SnC-targeting agent) can reduce the incidence of and/or risk of developing VCID by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can reduce the incidence of and/or risk of developing VCID in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat atherosclerosis by increasing healthspan and/or lifespan of a subject (e.g., a human subject or animal model) or a cell culture (e.g., a culture generated from a patient sample, or a repository of patient samples). A SnC-targeting agent (e.g., angptl2⁺ SnC-targeting agent) can increase healthspan and/or lifespan by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase healthspan and/or lifespan in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat atherosclerosis by reducing one or more symptoms or indications of atherosclerosis (e.g., one or more disease indications described in the aforementioned sections) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce one or more symptoms or indications of atherosclerosis by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can reduce one or more symptoms or indications of atherosclerosis in a subject or cell culture between 5-20%, between 5-50%, between 10-50%, between 10-80%, between 20-80%, or between 20-100%.

(ii) Treating Hepatic Steatosis

In specific embodiments, the methods and compositions described herein is used to target a SnC (e.g., an angptl2⁺ SnC, such as angptl2⁺ senescent hepatocyte and/or angptl2⁺ SnEC) for treating hepatic steatosis (e.g., NASH) in a subject (e.g., a human, such as a patient with hepatic steatosis) by administering to the subject an effective amount of a SnC-targeting agent (e.g., an angptl2⁺ SnC-targeting agent, such as shAngptl2) described herein. The method may include administering locally or systemically to the subject a SnC-targeting agent (e.g., an angptl2⁺ SnC-targeting agent) described herein in a dose (e.g., effective amount) and for a time sufficient to treat the hepatic steatosis.

The SnC-targeting agents (e.g., angptl2⁺ SnC-targeting agent, such as shAngptl2) can treat hepatic steatosis by inducing or increasing cell death (e.g., apoptosis, autophagy, and/or necroptosis) of one or more SnCs (e.g., angptl2⁺ SnCs, such as angptl2⁺ senescent hepatocyte and/or angptl2+ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase cell death (e.g., apoptosis, autophagy, and/or necroptosis) of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can induce or increase cell death (e.g., apoptosis, autophagy, and/or necroptosis) of SnCs (e.g., angptl2⁺ SnCs) in a subject or cell culture between 5-20%, between 5-50%, between 10-50%, between 20-80%, between 20-100%, between 50-200%, or between 100%-500%.

In particular, the SnC-targeting agents (e.g., angptl2⁺ SnC-targeting agent, such as shAngptl2) can treat hepatic steatosis (e.g., NASH) by inducing or increasing apoptosis of one or more SnCs (e.g., angptl2⁺ SnCs, such as angptl2⁺ senescent hepatocyte and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase apoptosis of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can induce or increase apoptosis of SnCs (e.g., angptl2+ SnCs) in a subject or cell culture between 5-20%, between 5-50%, between 10-50%, between 20-80%, between 20-100%, between 50-200%, or between 100%-500%.

In some embodiments, the SnC-targeting agents (e.g., angptl2⁺ SnC-targeting agent, such as shAngptl2) can treat hepatic steatosis (e.g., NASH) by inducing or increasing autophagy of one or more SnCs (e.g., angptl2⁺ SnCs, such as angptl2⁺ senescent hepatocyte and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase autophagy of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can induce or increase autophagy of SnCs (e.g., angptl2+ SnCs) in a subject or cell culture between 5-20%, between 5-50%, between 10-50%, between 20-80%, between 20-100%, between 50-200%, or between 100%-500%.

In some embodiments, the SnC-targeting agents (e.g., angptl2⁺ SnC-targeting agent, such as shAngptl2) can treat hepatic steatosis (e.g., NASH) by inducing or increasing necroptosis of one or more SnCs (e.g., angptl2⁺ SnCs, such as angptl2⁺ senescent hepatocyte and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can induce or increase necroptosis of SnCs (e.g., angptl2⁺ SnCs) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can induce or increase necroptosis of SnCs (e.g., angptl2+ SnCs) in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat hepatic steatosis (e.g., NASH) by reducing expression (e.g., mRNA and/or protein expression) of angptl2 (e.g., expression of angptl2 in a SnC, such as an angptl2⁺ SnC (e.g., angptl2⁺ senescent hepatocyte and/or angptl2⁺ SnEC)) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce expression (e.g., mRNA and/or protein expression) of angptl2 (e.g., expression of angptl2 in a SnC, such as an angptl2⁺ SnC) by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce expression (e.g., mRNA and/or protein expression) of angptl2 (e.g., expression of angptl2 in a SnC, such as an angptl2⁺ SnC) in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat hepatic steatosis (e.g., NASH) by reducing expression (e.g., mRNA and/or protein expression) and/or release of one or more SASP factors, such as inflammatory cytokines or chemokines (e.g., angptl2, IL-1β, IL-12, MCP-1, ICAM-1, TNF-α, IL-8, and/or IL-6) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce expression (e.g., mRNA and/or protein expression) and/or release of one or more SASP factors by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce expression (e.g., mRNA and/or protein expression) and/or release of one or more SASP factors in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat hepatic steatosis (e.g., NASH) by reducing inflammation (e.g., reducing expression (e.g., mRNA and/or protein expression) of one or more inflammatory mediators) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce inflammation by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce inflammation in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat hepatic steatosis (e.g., NASH) by reducing senescence (e.g., reducing expression (e.g., mRNA and/or protein expression) of one or more senescence mediators and/or senescence markers (e.g., p21, p53, or p16)) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce senescence by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can reduce senescence in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat hepatic steatosis (e.g., NASH) by reducing accumulation of SnC (e.g., angptl2⁺ SnC, such as angptl2+ senescent hepatocyte and/or angptl2⁺ SnEC) or increasing clearance of SnC (e.g., angptl2⁺ SnC, such as angptl2⁺ senescent hepatocyte and/or angptl2⁺ SnEC) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce accumulation of SnC or increase clearance of SnC by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce accumulation of SnC in a subject or cell culture between 5-20%, between 5-50%, between 10-50%, between 10-80%, between 20-80%, or between 20-100%. A SnC-targeting agent (e.g., angptl2⁺ SnC-targeting agent) can increase clearance of SnC in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat hepatic steatosis (e.g., NASH) by increasing immune surveillance (e.g., leading to increased clearance and/or reduced accumulation of SnC (e.g., angptl2⁺ SnC, such as angptl2⁺ senescent hepatocyte and/or angptl2⁺ SnEC)) in a subject (e.g., a human subject or animal model) or in a cell culture (e.g., a culture generated from a patient sample, or a repository of patient samples). A SnC-targeting agent (e.g., angptl2⁺ SnC-targeting agent) can increase immune surveillance by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase immune surveillance in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat hepatic steatosis (e.g., NASH) by increasing endothelial function in a subject (e.g., a human subject or animal model) or in a cell culture (e.g., a culture generated from a patient sample, or a repository of patient samples). A SnC-targeting agent (e.g., angptl2⁺ SnC-targeting agent) can increase endothelial function by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase endothelial function in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat hepatic steatosis (e.g., NASH) by increasing endothelial repair in a subject (e.g., a human subject or animal model) or in a cell culture (e.g., a culture generated from a patient sample, or a repository of patient samples). A SnC-targeting agent (e.g., angptl2⁺ SnC-targeting agent) can increase endothelial repair by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase endothelial repair in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat hepatic steatosis (e.g., NASH) by increasing hepatocyte repair in a subject (e.g., a human subject or animal model) or in a cell culture (e.g., a culture generated from a patient sample, or a repository of patient samples). A SnC-targeting agent (e.g., angptl2⁺ SnC-targeting agent) can increase hepatocyte repair by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase hepatocyte repair in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat hepatic steatosis (e.g., NASH) by reducing liver triglyceride level in a subject (e.g., a human subject or animal model) or in a cell culture (e.g., a culture generated from a patient sample, or a repository of patient samples). A SnC-targeting agent (e.g., angptl2⁺ SnC-targeting agent) can reduce liver triglyceride level by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce liver triglyceride level in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat hepatic steatosis (e.g., NASH) by reducing size of adipocytes in a subject (e.g., a human subject or animal model) or in a cell culture (e.g., a culture generated from a patient sample, or a repository of patient samples). A SnC-targeting agent (e.g., angptl2⁺ SnC-targeting agent) can reduce size of adipocytes by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce size of adipocytes in a subject or cell culture between 5-20%, between 5-50%, between 10-50%, between 10-80%, between 20-80%, or between 20-100%.

The SnC-targeting agents (e.g., angptl2⁺ SnC-targeting agent, such as shAngptl2) can treat hepatic steatosis (e.g., NASH) by increasing healthspan and/or lifespan of a subject (e.g., a human subject or animal model) or a cell culture (e.g., a culture generated from a patient sample, or a repository of patient samples). A SnC-targeting agent (e.g., angptl2⁺ SnC-targeting agent) can increase healthspan and/or lifespan by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can increase healthspan and/or lifespan in a subject or cell culture between 5-20%, between 5-50%, between 10-50%, between 20-80%, between 20-100%, between 50-200%, or between 100%-500%.

The SnC-targeting agents (e.g., angptl2⁺ SnC-targeting agent, such as shAngptl2) can treat hepatic steatosis (e.g., NASH) by reducing one or more symptoms or indications of hepatic steatosis (e.g., one or more disease indications described in the aforementioned sections) in a subject (e.g., a human subject or animal model) or in a cell culture (e.g., a culture generated from a patient sample, or a repository of patient samples). A SnC-targeting agent (e.g., angptl2⁺ SnC-targeting agent) can reduce one or more symptoms or indications of hepatic steatosis by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2+ SnC-targeting agent) can reduce one or more symptoms or indications of hepatic steatosis in a subject or cell culture between 5-20%, between 5-50%, between 10-50%, between 10-80%, between 20-80%, or between 20-100%.

(iii) Treating VCID

In specific embodiments, the methods and compositions described herein is used to target a SnC (e.g., an angptl2⁺ SnC, such as angptl2⁺ senescent brain cell and/or angptl2⁺ SnEC) for treating VCID in a subject (e.g., a human, such as a patient with VCID) by administering to the subject an effective amount of a SnC-targeting agent (e.g., an angptl2⁺ SnC-targeting agent, such as shAngptl2) described herein. The method may include administering locally or systemically to the subject a SnC-targeting agent (e.g., an angptl2⁺ SnC-targeting agent) described herein in a dose (e.g., effective amount) and for a time sufficient to treat the VCID.

The SnC-targeting agents (e.g., angptl2⁺ SnC-targeting agent, such as shAngptl2) can treat VCID by reducing one or more symptoms or indications of VCID in a subject (e.g., a human subject or animal model) or in a cell culture (e.g., a culture generated from a patient sample, or a repository of patient samples). A SnC-targeting agent (e.g., angptl2⁺ SnC-targeting agent) can reduce one or more symptoms or indications of VCID by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce one or more symptoms or indications of VCID in a subject or cell culture between 5-20%, between 5-50%, between 10-50%, between 10-80%, between 20-80%, or between 20-100%.

(iv) Treating an Autoimmune Disease

In specific embodiments, the methods and compositions described herein is used to target a SnC (e.g., an angptl2⁺ SnC, such as angptl2⁺ SnEC) for treating an autoimmune disease (e.g., arthritis or psoriasis) in a subject (e.g., a human, such as a patient with an autoimmune disease) by administering to the subject an effective amount of a SnC-targeting agent (e.g., an angptl2⁺ SnC-targeting agent, such as shAngptl2) described herein. The method may include administering locally or systemically to the subject a SnC-targeting agent (e.g., an angptl2⁺ SnC-targeting agent) described herein in a dose (e.g., effective amount) and for a time sufficient to treat the autoimmune disease.

The SnC-targeting agents (e.g., angptl2⁺ SnC-targeting agent, such as shAngptl2) can treat an autoimmune disease (e.g., arthritis or psoriasis) by reducing one or more symptoms or indications of the autoimmune disease in a subject (e.g., a human subject or animal model) or in a cell culture (e.g., a culture generated from a patient sample, or a repository of patient samples). A SnC-targeting agent (e.g., angptl2⁺ SnC-targeting agent) can reduce one or more symptoms or indications of the autoimmune disease by at least 1%, 2%, 5%, 10%, 15%, 20%, 25%, 30%, 35%, 40%, 45%, 50%, 55%, 60%, 65%, 70%, 75%, 80%, 85%, 90%, 95% or more compared to before administration to a subject or cell culture. A SnC-targeting agent (e.g., angptl2⁺ SnC-targeting agent) can reduce one or more symptoms or indications of the autoimmune disease in a subject or cell culture between 5-20%, between 5-50%, between 10-50%, between 10-80%, between 20-80%, or between 20-100%.

### Combination Therapies

In some embodiments, the SnC-targeting agents (e.g., angptl2⁺ SnC-targeting agent, such as shAngptl2) may be administered to a subject (e.g., a human, such as a human with a SnC-associated disease, such as an angptl2⁺ SnC-associated disease) in combination with a second therapeutic agent. The second therapeutic agent that is administered in combination with the SnC-targeting agent (e.g., angptl2⁺ SnC-targeting agent, such as shAngptl2) for treatment of a SnC-associated disease (e.g., an angptl2⁺ SnC-associated disease) may be one or more therapies that have been approved and/or routinely used (e.g., by clinicians) for treatment of that SnC-associated disease. For example, for treatment of atherosclerosis, a SnC-targeting agent (e.g., angptl2⁺ SnC-targeting agent, such as shAngptl2) is administered to a subject (e.g., a human, such as a human with atherosclerosis) in combination with a second therapeutic agent, where the second therapeutic agent is one or more therapies that have been approved and/or routinely used (e.g., by clinicians) for treatment of atherosclerosis, such as cholesterol lowering agents (e.g., statins, such as atorvastatin, fluvastatin, lovastatin, pitavastatin, pravastatin, rosuvastatin calcium, or simvastatin) and/or antihypertensive agents (e.g., diuretics, beta-blockers, ACE inhibitors, angiotensin II receptor blockers, calcium channel blockers, alpha blockers, alpha-2 receptor agonists, combined alpha and beta-blockers, central agonists, peripheral adrenergic inhibitors, or vasodilators). Alternatively, for treatment of hepatic steatosis (e.g., NASH), a SnC-targeting agent (e.g., angptl2⁺ SnC-targeting agent, such as shAngptl2) is administered to a subject (e.g., a human, such as a human with hepatic steatosis) in combination with a second therapeutic agent, where the second therapeutic agent is one or more therapies that have been approved and/or routinely used (e.g., by clinicians) for treatment of hepatic steatosis. Alternatively, for treatment of VCID, a SnC-targeting agent (e.g., angptl2⁺ SnC-targeting agent, such as shAngptl2) is administered to a subject (e.g., a human, such as a human with VCID) in combination with a second therapeutic agent, where the second therapeutic agent is one or more therapies that have been approved and/or routinely used (e.g., by clinicians) for treatment of VCID. For treatment of an autoimmune disease (e.g., arthritis or psoriasis), a SnC-targeting agent (e.g., angptl2⁺ SnC-targeting agent, such as shAngptl2) is administered to a subject (e.g., a human, such as a human with an autoimmune disease) in combination with a second therapeutic agent, where the second therapeutic agent is one or more therapies that have been approved and/or routinely used (e.g., by clinicians) for treatment of the autoimmune disease, such as immunosuppressive agents (e.g., corticosteroids, Janus kinase (JAK) inhibitors, calcineurin inhibitors, mTOR inhibitors, IMDH inhibitors, biologics, or monoclonal antibodies).

### Formulations and Carriers

In order to be administered to a subject, a pharmaceutical composition of the SnC-targeting agent (e.g., angptl2⁺ SnC-targeting agent, such as shAngptl2) either alone or in combination with one or more additional therapies (e.g., a second therapeutic agent) is formulated with a pharmaceutically acceptable carrier or excipient. A pharmaceutically acceptable carrier or excipient refers to a carrier (e.g., carrier, media, diluent, solvent, vehicle, etc.) which does not significantly interfere with the biological activity or effectiveness of the active ingredient(s) of a pharmaceutical composition and which is not excessively toxic to the host at the concentrations at which it is used or administered. Other pharmaceutically acceptable ingredients is present in the composition as well. Suitable substances and their use for the formulation of pharmaceutically active compounds are well-known in the art (see, for example, Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. For oral administration, agents is formulated by combining the active compounds with pharmaceutically acceptable carriers well known in the art. Such carriers enable the compounds of the invention to be formulated as a powder, tablet, pill, capsule, lozenge, liquid, gel, syrup, slurry, suspension, and the like. It is recognized that some pharmaceutical compositions, if administered orally, must be protected from digestion. This is typically accomplished either by complexing the protein with a composition to render it resistant to acidic and enzymatic hydrolysis or by packaging the protein in an appropriately resistant carrier such as a liposome. Suitable excipients for oral dosage forms include, for example, fillers such as sugars, including lactose, sucrose, mannitol, or sorbitol; cellulose preparations such as, for example, starch, gelatin, gum tragacanth, methyl cellulose, hydroxypropylmethyl cellulose, sodium carboxymethylcellulose, and/or polyvinylpyrrolidone (PVP). Disintegrating agents may be added, for example, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. For administration by inhalation, pharmaceutical compositions of this invention may be formulated in the form of an aerosol spray from a pressured container or dispenser, which contains a suitable propellant, e.g., a gas such as carbon dioxide, a fluorocarbon, or a nebulizer. Liquid or dry aerosol (e.g., dry powders, large porous particles, etc.) can also be used. For topical application, a pharmaceutical composition may be formulated in a suitable ointment, lotion, gel, or cream containing the active components suspended or dissolved in one or more pharmaceutically acceptable carriers suitable for use in such compositions.

Pharmaceutical compositions of the invention is administered parenterally in the form of an injectable formulation. Pharmaceutical compositions for injection is formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see, e.g., Banga (ed.) Therapeutic Peptides and Proteins: Formulation, Processing and Delivery Systems (3rd ed.) Taylor & Francis Group, CRC Press (2015). Pharmaceutical compositions may be prepared in microcapsules, such as hydroxylmethylcellulose or gelatin-microcapsule and poly-(methylmethacrylate) microcapsule. Pharmaceutical compositions may also be prepared in other drug delivery systems such as liposomes, albumin microspheres, microemulsions, nano-particles, and nanocapsules. Such techniques are described in Remington: The Science and Practice of Pharmacy 22^{th} edition (2012). The pharmaceutical compositions to be used for in vivo administration must be sterile. This is readily accomplished by filtration through sterile filtration membranes.

### Dosage and Routes of Administration

A SnC-targeting agent (e.g., angptl2⁺ SnC-targeting agent, such as shAngptl2) either alone or in combination with one or more additional therapies (e.g., a second therapeutic agent) is administered as a pharmaceutical composition to a subject (e.g., a human, such as a human with a SnC-associated disease, such as an angptl2⁺ SnC-associated disease) in a variety of ways. The composition must be suitable for the subject receiving the treatment, and the mode of administration. The composition used in this invention is administered orally, sublingually, parenterally, intravenously, subcutaneously, intramedullary, intranasally, as a suppository, using a flash formulation, topically, intradermally, subcutaneously, via pulmonary delivery, via intra-arterial injection, or via a mucosal route. In specific embodiments, a SnC-targeting agent (e.g., angptl2⁺ SnC-targeting agent, such as shAngptl2) either alone or in combination with one or more additional therapies (e.g., a second therapeutic agent) is administered as a pharmaceutical composition to a subject (e.g., a human, such as a human with a SnC-associated disease, such as an angptl2⁺ SnC-associated disease) systemically (e.g., intravenously) or locally (e.g., at the disease site). Such local administrations may restrict the effect of the treatment to the site of administration.

The dosage of the pharmaceutical compositions of the SnC-targeting agent (e.g., angptl2+ SnC-targeting agent, such as shAngptl2) either alone or in combination with one or more additional therapies (e.g., a second therapeutic agent) depends on factors including the route of administration, the severity of the condition to be treated, and physical characteristics, e.g., age, weight, general health, of the subject. A pharmaceutical composition may include a dosage ranging from 1 ng/kg to about 100 g/kg (e.g. 1-10 ng/kg, e.g, 2 ng/kg, 3 ng/kg, 4 ng/kg, 5 ng/kg, 6 ng/kg, 7 ng/kg, 8 ng/kg, 9 ng/kg, 10 ng/kg, e.g., 10-100 ng/kg, e.g., 20 ng/kg, 30 ng/kg, 40 ng/kg, 50 ng/kg, 60 ng/kg, 70 ng/kg, 80 ng/kg, 90 ng/kg, 100 ng/kg, e.g., 100-1 μg/kg, e.g., 200 ng/kg, 300 ng/kg, 400 ng/kg, 500 ng/kg, 600 ng/kg, 700 ng/kg, 800 ng/kg, 900 ng/kg, 1 μg/kg, e.g. 1-10 μg/kg, e.g. 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, e.g., 10-100 μg/kg, e.g., 20 μg/kg, 30 μg/kg, 40 μg/kg, 50 μg/kg, 60 μg/kg, 70 μg/kg, 80 μg/kg, 90 μg/kg, 100 μg/kg, e.g., 100 μg/kg-1 mg/kg, e.g., 200 μg/kg, 300 μg/kg, 400 μg/kg, 500 μg/kg, 600 μg/kg, 700 μg/kg, 800 μg/kg, 900 μg/kg, 1 mg/kg, e.g., 1-10 mg/kg, e.g., 2 mg/kg, 3 mg/kg, 4 mg/kg, 5 mg/kg, 6 mg/kg, 7 mg/kg, 8 mg/kg, 9 mg/kg, 10 mg/kg, e.g. 10-100 mg/kg, e.g., 20 mg/kg, 30 mg/kg, 40 mg/kg, 50 mg/kg, 60 mg/kg, 70 mg/kg, 80 mg/kg, 90 mg/kg, 100 mg/kg, e.g., 100-1 g/kg, e.g., 200 mg/kg, 300 mg/kg, 400 mg/kg, 500 mg/kg, 600 mg/kg, 700 mg/kg, 800 mg/kg, 900 mg/kg, 1 g/kg, e.g., 1-10 g/kg, e.g. 2 g/kg, 3 g/kg, 4 g/kg, 5 g/kg, 6 g/kg, 7 g/kg, 8 g/kg, 9 g/kg, 10 g/kg, e.g., 10-100 g/kg, e.g., 20 g/kg, 30 g/kg, 40 g/kg, 50 g/kg, 60 g/kg, 70 g/kg, 80 g/kg, 90 g/kg, 100 g/kg).

The dosage regimen may be determined by the clinical indication being addressed, as well as by various variables (e.g. weight, age, sex of subject) and clinical presentation (e.g. extent or severity of condition). Furthermore, it is understood that all dosages may be continuously given or divided into dosages given per a given time frame. Pharmaceutical compositions that include the SnC-targeting agent (e.g., angptl2+ SnC-targeting agent, such as shAngptl2) either alone or in combination with one or more additional therapies (e.g., a second therapeutic agent) may be administered to a subject in need thereof, for example, one or more times (e.g., 1-10 times or more) daily, weekly, biweekly, monthly, bimonthly, quarterly, biannually, annually, or as medically necessary. Dosages may be provided in either a single or multiple dosage regimens. The timing between administrations may decrease as the medical condition improves or increase as the health of the patient declines.

### Modalities of SnC-Targeting Agents

The SnC-targeting agent (e.g., an angptl2⁺ SnC-targeting agent, such as an angptl2⁺ SnEC-targeting agent) that is used for targeting a SnC (e.g., an angptl2⁺ SnC, such as an angptl2⁺ SnEC) in one or more methods described herein is selected from different modalities.

### Inhibitory RNA Molecule

The SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) that is used for targeting a SnC (e.g., an angptl2⁺ SnC, such as an angptl2⁺ SnEC, an angptl2⁺ senescent hepatocyte, an angptl2⁺ senescent cardiac cell, an angptl2⁺ senescent brain cell, an angptl2⁺ senescent glial cell, an angptl2⁺ senescent neuron, or an angptl2⁺ senescent synovial cell) in one or more methods described herein is a nucleic acid molecule, such as an inhibitory RNA molecule (e.g., a short hairpin RNA (shRNA), a short interfering RNA (siRNA), and/or a microRNA (miRNA) molecule). A siRNA is a double-stranded RNA molecule that typically has a length of about 19-25 base pairs. A shRNAs is delivered to cells in the form of plasmids, (e.g., viral or bacterial vectors) by transfection, electroporation, or transduction. A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA.

Specifically, the SnC-targeting agent (e.g., an angptl2⁺ SnC-targeting agent, such as an angptl2⁺ SnEC-targeting agent, an angptl2⁺ senescent hepatocyte-targeting agent, an angptl2+ senescent cardiac cell-targeting agent, an angptl2⁺ senescent brain cell-targeting agent, an angptl2+ senescent glial cell-targeting agent, an angptl2⁺ senescent neuron-targeting agent, or an angptl2+ senescent synovial cell-targeting agent) that is used for targeting a SnC (e.g., an angptl2⁺ SnC, such as an angptl2⁺ SnEC, an angptl2⁺ senescent hepatocyte, an angptl2⁺ senescent cardiac cell, an angptl2⁺ senescent brain cell, an angptl2⁺ senescent glial cell, an angptl2⁺ senescent neuron, or an angptl2⁺ senescent synovial cell) in one or more methods described herein is a shRNA (e.g., shAngptl2). In particular, a shRNA (e.g., shAngptl2) targeting a SnC (e.g., an angptl2⁺ SnC, such as an angptl2⁺ SnEC) is used in the methods described herein to: (i) reduce the expression (e.g., mRNA and/or protein expression) of angptl2; (ii) induce cell death (e.g., apoptosis, autophagy, and/or necroptosis) of a SnC (e.g., an angptl2⁺ SnC, such as an angptl2⁺ SnEC); and/or (iii) treat a SnC-associated disease (e.g., an angptl2⁺ SnC-associated disease, such as an angptl2⁺ SnEC-associated disease). The shRNA (e.g., shAngptl2) that is used to target a SnC (e.g., an angptl2⁺ SnC, such as an angptl2⁺ SnEC) in the methods described herein is delivered to a cell (e.g., a SnC (e.g., an angptl2⁺ SnC, such as an angptl2⁺ SnEC)) in the form of plasmids (e.g., one or more plasmids from Table 1).

The SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) that is used for targeting a SnC (e.g., an angptl2⁺ SnC, such as an angptl2⁺ SnEC, an angptl2⁺ senescent hepatocyte, an angptl2⁺ senescent cardiac cell, an angptl2⁺ senescent brain cell, an angptl2⁺ senescent glial cell, an angptl2⁺ senescent neuron, or an angptl2⁺ senescent synovial cell) in one or more methods described herein is a siRNA. In some embodiments, a siRNA targeting a SnC (e.g., an angptl2⁺ SnC, such as an angptl2⁺ SnEC) is used in the methods described herein to: (i) reduce the expression (e.g., mRNA and/or protein expression) of angptl2; (ii) induce cell death (e.g., apoptosis, autophagy, and/or necroptosis) of a SnC (e.g., an angptl2⁺ SnC, such as an angptl2+ SnEC); and/or (iii) treat a SnC-associated disease (e.g., an angptl2⁺ SnC-associated disease, such as an angptl2⁺ SnEC-associated disease).

The SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) that is used for targeting a SnC (e.g., an angptl2⁺ SnC, such as an angptl2⁺ SnEC, an angptl2⁺ senescent hepatocyte, an angptl2⁺ senescent cardiac cell, an angptl2⁺ senescent brain cell, an angptl2⁺ senescent glial cell, an angptl2⁺ senescent neuron, or an angptl2⁺ senescent synovial cell) in one or more methods described herein is a miRNA. In some embodiments, a miRNA targeting a SnC (e.g., an angptl2⁺ SnC, such as an angptl2⁺ SnEC) is used in the methods described herein to: (i) reduce the expression (e.g., mRNA and/or protein expression) of angptl2; (ii) induce cell death (e.g., apoptosis, autophagy, and/or necroptosis) of a SnC (e.g., an angptl2⁺ SnC, such as an angptl2+ SnEC); and/or (iii) treat a SnC-associated disease (e.g., an angptl2⁺ SnC-associated disease, such as an angptl2⁺ SnEC-associated disease).

In some embodiments, a viral vector (e.g., adeno-associated virus serotype 1 (AAV1), adeno-associated virus serotype 8 (AAV8), or adeno-associated virus serotype 9 (AAV9)) containing a plasmid (e.g., one or more plasmids from Table 1) is used to encode a SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) for use in one or more methods described herein. In particular, an AAV1, AAV8, or AAV9 containing a plasmid (e.g., one or more plasmids from Table 1) is used to encode a SnC-targeting shRNA (e.g., shAngptl2) for use in one or more methods described herein. A SnC-targeting shRNA (e.g., shAngptl2) encoded by a plasmid (e.g., one or more plasmids from Table 1) that is carried in a viral vector (e.g., AAV1 and AAV9 for vascular targeting or AAV8 for liver targeting) is used in one or more methods described herein to: (i) reduce the expression (e.g., mRNA and/or protein expression) of angptl2; (ii) induce cell death (e.g., apoptosis) of a SnC (e.g., an angptl2⁺ SnC, such as an angptl2⁺ SnEC); and/or (iii) treat a SnC-associated disease (e.g., an angptl2⁺ SnC-associated disease, such as an angptl2⁺ SnEC-associated disease).

Examples of viral vectors include a retrovirus (e.g., Retroviridae family viral vector), adenovirus (e.g., Ad5, Ad26, Ad34, Ad35, and Ad48), parvovirus (e.g., AAV, such as AAV1, AAV8 and AAV9), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030.

An inhibitory RNA molecule for use in one or more methods described herein is modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity. The making and use of inhibitory agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010.

The synthetic mRNA molecule for use in one or more methods described herein is modified, e.g., chemically. The mRNA molecule is chemically synthesized or transcribed in vitro. The mRNA molecule is disposed on a plasmid, e.g., a viral vector, bacterial vector, or eukaryotic expression vector. In some examples, the mRNA molecule is delivered to cells by transfection, electroporation, or transduction (e.g., adenoviral or lentiviral transduction).

Methods of making and purifying modified RNAs are known and disclosed in the art. For example, modified RNAs are made using only in vitro transcription (IVT) enzymatic synthesis. Methods of making IVT polynucleotides are known in the art and are described in e.g., WO2013151666, WO2013151668, WO2013151663, WO2013151669, WO2013151670, WO2013151664, WO2013151665, WO2013151671, WO2013151672, WO2013151667, and WO2013151736. Methods of purification include purifying an RNA transcript containing a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO2014152031); using ion (e.g., anion) exchange chromatography that allows for separation of longer RNAs up to 10,000 nucleotides in length via a scalable method (WO2014144767); and subjecting a modified mRNA sample to DNAse treatment (WO2014152030).

Formulations of modified RNAs are known and are described, e.g., in WO2013090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs), and combinations thereof.

Modified RNAs encoding polypeptides in the fields of human disease, antibodies, viruses, and a variety of in vivo settings are known and are disclosed in for example, Table 6 of International Publication Nos. WO2013151666, WO2013151668, WO2013151663, WO2013151669, WO2013151670, WO2013151664, WO2013151665, and WO2013151736; Tables 6 and 7 of International Publication No. WO2013151672; Tables 6 and 178 of International Publication No. WO2013151671; Tables 6, 185 and 186 of International Publication No. WO2013151667. Any of the foregoing may be synthesized as an IVT polynucleotide, chimeric polynucleotide or a circular polynucleotide, and each may comprise one or more modified nucleotides or terminal modifications.

### Polypeptides

The SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) that is used for targeting a SnC (e.g., an angptl2⁺ SnC, such as an angptl2⁺ SnEC, an angptl2⁺ senescent hepatocyte, an angptl2⁺ senescent cardiac cell, an angptl2⁺ senescent brain cell, an angptl2⁺ senescent glial cell, an angptl2⁺ senescent neuron, or an angptl2⁺ senescent synovial cell) in one or more methods described herein is a polypeptide (e.g., an inhibitory antibody, or a targeting antibody). In some embodiments, an inhibitory antibody, or a targeting antibody targeting a SnC (e.g., an angptl2⁺ SnC, such as an angptl2⁺ SnEC) is used in the methods described herein to: (i) reduce the expression (e.g., mRNA and/or protein expression) of angptl2; (ii) induce death (e.g., apoptosis) of a SnC (e.g., an angptl2⁺ SnC, such as an angptl2⁺ SnEC); and/or (iii) treat a SnC-associated disease (e.g., an angptl2⁺ SnC-associated disease, such as an angptl2⁺ SnEC-associated disease).

Polypeptides is produced by a variety of recombinant and synthetic techniques, such as by recombinant gene expression or solid-phase peptide synthesis procedures known in the art. For instance, one of skill in the art can design polynucleotides encoding, e.g., two or more CDRs operably linked to one another in frame so as to produce a continuous, single-chain peptide containing these CDRs. Optionally, the CDRs may be separated by a spacer, such as by a framework region (e.g., a framework region of a germline consensus sequence of a human antibody) or a flexible linker, such as a poly-glycine or glycine/serine linker known in the art. When produced by chemical synthesis methods, native chemical ligation can optionally be used as a strategy for the synthesis of long peptides (e.g., greater than 50 amino acids). Native chemical ligation protocols are known in the art and have been described, e.g., by Dawson et al. (Science, 266:776-779, 1994); incorporated herein by reference.

Methods of making a polypeptide are routine in the art. See, in general, Smales & James (Eds.), Therapeutic Proteins: Methods and Protocols (Methods in Molecular Biology), Humana Press 2005; and Crommelin, Sindelar & Meibohm (Eds.), Pharmaceutical Biotechnology: Fundamentals and Applications, Springer 2013. Some methods for producing a polypeptide involve expression in mammalian cells, although recombinant proteins can also be produced using insect cells, yeast, bacteria, or other cells under the control of appropriate promoters. Mammalian expression vectors may contain non-transcribed elements such as an origin of replication, a suitable promoter and enhancer, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press 2012.

Various mammalian cell culture systems is employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer 2014.

### Small Molecule

The SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) that is used for targeting a SnC (e.g., an angptl2⁺ SnC, such as an angptl2⁺ SnEC, an angptl2⁺ senescent hepatocyte, an angptl2⁺ senescent cardiac cell, an angptl2⁺ senescent brain cell, an angptl2⁺ senescent glial cell, an angptl2⁺ senescent neuron, or an angptl2⁺ senescent synovial cell) in one or more methods described herein is a small molecule (e.g., a small molecule inhibitor). In some embodiments, a small molecule inhibitor targeting a SnC (e.g., an angptl2⁺ SnC, such as an angptl2⁺ SnEC) is used in the methods described herein to: (i) reduce the expression (e.g., mRNA and/or protein expression) of angptl2; (ii) induce cell death (e.g., apoptosis, autophagy, and/or necroptosis) of a SnC (e.g., an angptl2⁺ SnC, such as an angptl2⁺ SnEC); and/or (iii) treat a SnC-associated disease (e.g., an angptl2⁺ SnC-associated disease, such as an angptl2⁺ SnEC-associated disease).

A pharmaceutical composition comprising one or more of the small molecules is formulated for treatment of a SnC-associated disease, such as an angptl2⁺ SnC-associated disease in a subject (e.g., a human, such as a human patient in need thereof). In some embodiments, a pharmaceutical composition that includes the small molecule is formulated for local administration, e.g., to the affected site in a subject.

### Nuclease

The SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) that is used for targeting a SnC (e.g., an angptl2⁺ SnC, such as an angptl2⁺ SnEC, an angptl2⁺ senescent hepatocyte, an angptl2⁺ senescent cardiac cell, an angptl2⁺ senescent brain cell, an angptl2⁺ senescent glial cell, an angptl2⁺ senescent neuron, or an angptl2⁺ senescent synovial cell) in one or more methods described herein is a nuclease (e.g., Cas9, zinc finger nuclease (ZFN), or transcription activator-like effector-based nuclease (TALEN)). In some embodiments, a Cas9, TALEN, or ZFN targeting a SnC (e.g., an angptl2⁺ SnC, such as an angptl2⁺ SnEC) is used in the methods described herein to: (i) reduce the expression (e.g., mRNA and/or protein expression) of angptl2; (ii) induce cell death (e.g., apoptosis, autophagy, and/or necroptosis) of a SnC (e.g., an angptl2⁺ SnC, such as an angptl2⁺ SnEC); and/or (iii) treat a SnC-associated disease (e.g., an angptl2⁺ SnC-associated disease, such as an angptl2⁺ SnEC-associated disease).

The SnC-targeting agent (e.g., an angptl2⁺ SnC-targeting agent, such as an angptl2⁺ SnEC-targeting agent, an angptl2⁺ senescent hepatocyte-targeting agent, an angptl2⁺ senescent cardiac cell-targeting agent, an angptl2⁺ senescent brain cell-targeting agent, an angptl2⁺ senescent glial cell-targeting agent, an angptl2⁺ senescent neuron-targeting agent, or an angptl2⁺ senescent synovial cell-targeting agent) that is used for targeting a SnC (e.g., an angptl2⁺ SnC, such as an angptl2⁺ SnEC, an angptl2⁺ senescent hepatocyte, an angptl2⁺ senescent cardiac cell, an angptl2⁺ senescent brain cell, an angptl2⁺ senescent glial cell, an angptl2⁺ senescent neuron, or an angptl2⁺ senescent synovial cell) in one or more methods described herein is modified. For example, the modification is a chemical modification (e.g., conjugation to a marker, such as a fluorescent marker or a radioactive marker), conjugation to a molecule that enhances the stability or half-life of the agent, or conjugation to a targeting molecule to target the agent to a particular cell or tissue (e.g., bioconjugation to a targeting antibody to target the agent to a specific tissue (e.g., cardiac tissue or hepatic tissue).

Additionally, the modification is a packaging modification (e.g., packaging within a nanoparticle or microparticle), or targeting modification to prevent the agent from crossing the blood brain barrier.

### Examples

The following examples are put forth to provide those of ordinary skill in the art with a description of how the methods described herein may be used and evaluated, and are intended to be purely exemplary of the invention and are not intended to limit the scope of the claims.

### Example 1. Methods

Animal Study

All animal experiments were performed in accordance with the “Guide for the Care and Use of Experimental Animals of the Canadian Council on Animal Care” and the “Guide for the Care and Use of Laboratory Animals” of the US National Institutes of Health (NIH Publication No. 85-23, revised 1996) and was approved by the Montreal Heart Institute Ethics Committee. Knockout/transgenic severely dyslipidemic LDLr^{-/-}; hApoB^{+/+} (ATX) male mice were fed a normal diet, as described in Bolduc et al. (Am J Physiol Heart Circ Physiol, 301: H2081-2092, 2011) and Gendron et al. (Am J Physiol Heart Circ Physiol, 298: H2062-2070, 2010). Mice were anaesthetized with 44 mg/kg ketamine and 2.2 mg/kg xylazine, and ventilated. Thoracic aortas of ATX mice were used to quantify atherosclerotic lesions and to assess mRNA expression of senescent markers, inflammatory markers, progenitor marker and apoptosis markers, both in the freshly isolated native endothelium and in the aorta wall (Farhat et al., J Am Heart Assoc, 2: e000201, 2013). Hearts and plasma of ATX mice were used to validate the vascular tropism of the AAV1. Angptl2 knockdown (KD) male and wild-type (WT) male littermates used in this study were from our colony, and genotyped as described in Yu et al. (J Am Heart Assoc, 3: e001024, 2014). KD and WT littermate mice were used to assess lifespan.

### AAV1 Production

The protocol for adeno-associated virus serotype 1 (AAVI) production was adapted from Chen et al. (Hum Gene Ther, 16: 235-247, 2005). HEKT293 competent cells were plated until a confluence of 70%, and then transfected with 12 μg/mL of the pXYZ C1 plasmid vector (serotype 1), 4 μg/mL of the plasmid containing shAngptl2, shSCR (Table 1) or mCherry red fluorescent protein sequence and 48 μg/mL of polyethylenimin (SIGMA) overnight in a starvation medium. The next day, the medium was changed, and cells were incubated for 48 h. The cells were then collected and lysed with Tris 1M/NaCl 5M, pH 8.5. Lysis was boosted by several freeze/thaw steps, and 1 μL of MgCl₂ (1M) in addition to benzonase (250 U/μL, SIGMA) were added per mL of lysate. After centrifugation, AAV1 were isolated from the supernatant by iodixanol gradients during ultracentrifugation, extracted with a syringe and concentrated in a PBST buffer between 0.25 ml-0.5 ml in volume with Ultracel-100 regenerated cellulose membrane (100 kDa, EMD MILLIPORE).

### AAV1 Titration and Administration in ATX Mice

Titration was performed by qPCR reactions using a STEPONEPLUS Real-Time PCR System (THERMO FISHER SCIENTIFIC). AAV1 were quantified using a standard range made by serial dilutions (10 ng to 10⁻⁶ ng) of shAngptl2 and shSCR plasmids containing a target sequence (BGH) or the plasmid containing mCherry sequence. The primers for BGH target sequence and mCherry were designed using CLONE MANAGER software (Table 2). Each mouse received a systemic (intravenous, i.v.) bolus injection of 1011 AAV1 particles at 3-month old (-mo), and were studied 1 week, 2 weeks or 4 weeks after the injection, at 6-mo.

### Quantification of Atherosclerotic Lesions

Freshly isolated thoracic aortas of ATX mice were longitudinally opened and pictures taken with a digital camera. Hepatic steatosis lesions were quantified by measuring white spots in the aorta using ImageJ software, as described previously in Farhat et al. (J Am Heart Assoc, 2: e000201, 2013). White spots areas were expressed as percentage of total aorta area.

### Total RNA Extraction

Total RNA was extracted from freshly isolated aorta. Native endothelial cells (EC)s were scraped with a blade from longitudinally opened segments of the thoracic aorta (Farhat et al., J Am Heart Assoc, 2: e000201, 2013), and denuded aorta wall or total heart were pulverized in liquid nitrogen with a CELL CRUSHER kit (CELLCRUSHER LIMITED). RNA was extracted using an RNEASY Mini kit (QIAGEN). Contamination with genomic DNA was prevented by digestion with a DNASE I mix (QIAGEN) according to the manufacturer's guidelines. Total RNA was quantified using a NANODROP ND-100 spectrophotometer.

### Real-Time Quantitative PCR

Total RNA was reverse transcribed into first-strand complementary DNA with M-MLV reverse transcriptase (THERMO FISHER), using random hexamer primers. The qPCR reactions were carried out on diluted RT products by using the DNA-binding dye SYBR Green PCR Master Mix (THERMO FISHER SCIENTIFIC) to detect PCR products with STEPONEPLUS Real-Time PCR System (THERMO FISHER SCIENTIFIC). The primers of target genes (Angptl2, p21, Pai-1, II-1β, Icam-1, Cd68, Cd34, Mcp1, Bax, Bcl2) were designed using Clone Manager software (Table 2). All samples were run in duplicate and the fold changes in gene expression were calculated by a ΔΔC_{T} method using cycloA (cyclophilin A) as the housekeeping gene.

### Immunofluorescence

Fixed tissues (transversely aortic frozen sections) were incubated with 1:1000 diluted rabbit anti-mCherry (#ab167453; ABCAM) and 1:800 diluted secondary antibody Alexa fluor-647 anti-rabbit (#A31573, THERMO FISHER SCIENTIFIC). DNA counterstaining was performed by incubating fixed tissues with DAPI (D1306, THERMO FISHER SCIENTIFIC). Fluorescence was visualized by confocal microscopy (ZEISS LSM 510).

### Biochemical Analysis

Blood was rapidly collected after sacrifice and centrifuged within minutes to collect the plasma. Samples were stored frozen in aliquots at −80° C. until further analysis. Plasma metabolic profile (total cholesterol, glucose and triglycerides) was measured at the biochemical laboratory of the Montreal Heart Institute.

### Human Study

Twenty-six coronary artery disease (CAD) patients undergoing coronary arterial bypass (CABG, n=16), or both CABG and aortic or mitral valve replacement (VR) (n=10) between February 2014 and January 2016, were prospectively included in the study at the Montreal Heart Institute (Quebec, Canada). Inclusion criteria were patients undergoing elective CABG and VR with normal left ventricular function at pre-operative evaluation. Patients who underwent other type of procedure were excluded. All patients signed an informed consent and the study (#ICM 13-1492) was approved by the ethical committee on human research of the Montreal Heart Institute. On the overall population, the mean age was 67±8 years and 92% were male. All patients had at least one risk factor for CVD and received preventive treatment, such as aspirin or statin before surgery. Left ventricular function was normal in all patients. The basal pre-operative characteristics of the patients are detailed in the Table 3. All patients underwent cardiac surgery with cardiopulmonary bypass (CPB). During surgery, a distal segment of left internal mammary artery (IMA) measuring at least 1 cm in length was harvested and stored at −80° C. in all patients. Gene expression of ANGPTL2 and other markers of inflammation (TNFα, IL-8, IL-6) and senescence (p21) were measured in these tissues. Frozen IMA fragments were pulverized in liquid nitrogen and dry ice with a CELL CRUSHER kit (CELLCRUSHER LIMITED). Samples of organ powder were then processed for RNA extraction according to the manufacturer's instructions (RNEASY Mini kit, QIAGEN). Contamination with DNA was prevented by digestion with a DNASE I mix (QIAGEN) according to the manufacturer's guidelines. Total RNA was quantified using a NANODROP ND-100 spectrophotometer. Total RNA was reverse-transcribed into first-strand complementary DNA with M-MLV reverse transcriptase (THERMO FISHER SCIENTIFIC) using random hexamer primers. The primers of target genes were designed using CLONE MANAGER software (Table 4). Quantitative polymerase chain reaction was performed using the SYBR Green PCR Master Mix (THERMO FISHER SCIENTIFIC). All samples were run in duplicate and the fold changes in gene expression were calculated by a ΔΔCT method using GAPDH as the housekeeping gene. Data are presented as mean±SEM of n patients in the text and in the Table 3.

### Statistical Analysis

Data are presented as mean±SEM, with “n” indicating the number of animals or patients. Animal data were analyzed using parametric and non-parametric tests: the two-way unpaired t test or Mann Whitney test were used to compare mRNA expression in shSCR group (control) versus shAngptl2 group; 1-way ANOVA with Tukey's post-test was used to compare plaque area in untreated 3-mo, AAV1-treated or -untreated 6-mo ATX mice. A two-way unpaired t test was used to compare plaque growth with endothelial Angptl2 and p21 expression in ATX mice at 3-mo vs. 12-mo. Gehan-Breslow-Wilcoxon non-parametric tests were performed to compare health span of KID and WT littermate mice. Human data are presented as dot plots. Non-parametric Spearman correlation analyses were performed to compare the mRNA levels of ANGPTL2, TNFα, IL-8, IL-6 and p21 in IMA segments from patients. All statistical analyses were performed using Graph Pad Prism 5.0.

TABLE 1
shRNA sequences

| shRNA | sequence |
|---|---|
| Angptl2 | GCAGAGTCTTCCAATCAGTTAATCAAGAGTTAACT |
| | GATTGGAAGACTCTGC (SEQ ID NO: 1) |
| Scramble* | CCTAAGGTTAAGTCGCCCTCGCTCGAGCGAGGGCG |
| | ACTTAACCTTAGG (SEQ ID NO: 2) |

*The scramble sequence is directed against luciferase not expressed in mammals.

TABLE 2
Mouse primers used to quantify gene expression using quantitative RT-PCR

| Target gene | Forward | Reverse |
|---|---|---|
| Angptl2 | GATCCAGAGTGACCAGAATC | TCTCAGGCTTCACCAGGTAG |
| | (SEQ ID NO: 3 | (SEQ ID NO: 4) |
| p21 | TGTCGCTGTCTTGCACTCT (SEQ ID NO: 5) | AGACCAATCTGCGCTTGGA (SEQ |
| | | ID NO: 6) |
| Cd68 | CATCAGAGCCCGAGTACAGTCTACC (SEQ | AATTCTGCGCCATGAATGTCC |
| | ID NO: 7) | (SEQ ID NO: 8) |
| Cd34 | TGAGATGACATCACCCACCG (SEQ ID | GCCAACCTCACTTCTCGGAT (SEQ |
| | NO: 9) | ID NO: 10) |
| Icam-1 | CAATTCACACTGAATGCCAGCTC (SEQ ID | CAAGCAGTCCGTCTCGTCCA (SEQ |
| | NO: 11) | ID NO: 12) |
| II-1β | TGCCACCTTTTGACAGTGATG (SEQ ID | TGATGTGCTGCTGCGAGATT (SEQ |
| | NO: 13) | ID NO: 14) |
| Mcp1 | GCAGGTCCCTGTCATGCTTC (SEQ ID | CTCTCCAGCCTACTCATTGGG |
| | NO: 15) | (SEQ ID NO: 16) |
| CycloA | CCGATGACGAGCCCTTGG (SEQ ID NO: 17) | GCCGCCAGTGCCATTATG (SEQ ID |
| | | NO: 18) |
| BcI2 | GTGGTGGAGGAACTCTTCAG (SEQ ID | GTTCCACAAAGGCATCCCAG (SEQ |
| | NO: 19) | ID NO: 20) |
| Bax | AGCAAACTGGTGCTCAAGGC (SEQ ID | CCACAAAGATGGTCACTGTC (SEQ |
| | NO: 21) | ID NO: 22) |
| Mcherry | AGGTCAAGACCACCTAAAA (SEQ ID | CTGTTCCACGATGGTGTAGT (SEQ |
| | NO: 23) | ID NO: 24) |
| Bgh | TGCCTTCCTTGACCCT (SEQ ID NO: 25) | CCTTGCTGTCCTGCCC (SEQ ID |
| | | NO: 26) |
| Pai-1 | TTGTCCAGCGGGACCTAGAG (SEQ ID NO: | AAGTCCACCTGTTTCACCATAGTCT |
| | 39) | (SEQ ID NO: 40) |

Angptl2, angiopoietin-like 2;
Icam-1, intracellular adhesion molecule;
IL-1β, interleukin 1β;
Mcp1, Monocyte chemoattractant protein 1;
CycloA, cyclophilin A;
Pai-1, plasminogen activator inhibitor-1.

TABLE 3
Clinical profiles of the atherosclerotic patients undergoing
artery bypass graft surgery and/or valve replacement

| | | All CAD | | Valve + | | | |
|---|---|---|---|---|---|---|---|
| | | patients | | CABG | | CABG | |
| | | n = 26 | | n = 10 | | n = 16 | |
| Demographics | Age (years) | 67 ± 2 | | 69 ± 2 | | 65 ± 2 | |
| | Sex (men, %) | 24 | (92%) | 10 | (100%) | 14 | (88%) |
| | Height (m) | 1.71 ± 0.02 | | 1.68 ± 0.01 | | 1.73 ± 0.03 | |
| | Weight (kg) | 84.6 ± 2.5 | | 84.1 ± 3.8 | | 85.0 ± 3.5 | |
| | BMI (kg/m²) | 28.9 ± 0.8 | | 29.8 ± 2.0 | | 28.3 ± 0.9 | |
| Risk factors | Dyslipidemia | 24 | (92%) | 8 | (80%) | 16 | (100%) |
| (n, %) | Hypertension | 22 | (85%) | 9 | (90%) | 13 | (81%) |
| | Obesity | 12 | (46%) | 6 | (60%) | 6 | (38%) |
| | Diabetes | 11 | (42%) | 3 | (30%) | 8 | (50%) |
| | Active smoker | 3 | (12%) | 1 | (10%) | 2 | (12%) |
| | Ex-smoker | 9 | (35%) | 4 | (40%) | 5 | (31%) |
| | COPD | 2 | (8%) | 2 | (20%) | 0 | (0%) |
| | Family history of CVD | 9 | (35%) | 4 | (40%) | 5 | (31%) |
| Medication | β-blockers | 22 | (85%) | 8 | (80%) | 14 | (88%) |
| | ACE inhibitors | 4 | (15%) | 1 | (10%) | 3 | (29%) |
| | AR antagonists | 6 | (23%) | 3 | (30%) | 3 | (29%) |
| | Diuretic | 9 | (35%) | 3 | (30%) | 6 | (38%) |
| | Hypoglycemic agent | 9 | (35%) | 1 | (10%) | 8 | (50%) |
| | Anticoagulant | 4 | (15%) | 1 | (10%) | 3 | (19%) |
| | Aspirin | 24 | (92%) | 8 | (80%) | 16 | (100%) |
| | Statin | 25 | (96%) | 9 | (90%) | 16 | (100%) |

| | | | | |
|---|---|---|---|---|
| Blood analysis | Total cholesterol | 3.3 ± 0.2 | 3.3 ± 0.3 | 3.2 ± 0.3 |
| | (mmol/L) | | | |
| | Cholesterol-LDL | 1.5 ± 0.2 | 1.5 ± 0.2 | 1.5 ± 0.3 |
| | (mmol/L) | | | |
| | Cholesterol-HDL | 1.2 ± 0.07 | 1.0 ± 0.1 | 1.3 ± 0.09 |
| | (mmol/L) | | | |
| | Triglycerides (mmol/L) | 1.4 ± 0.1 | 1.7 ± 0.3 | 1.2 ± 0.1 |
| | HbA1C (%) | 0.064 ± 0.008 | 0.059 ± 0.03 | 0.066 ± 0.003 |
| | | (11) | (3) | (8) |

Data are mean ± SEM of (n) patients. No significant difference between groups (One-way ANOVA followed by Tukey's post hoc test, or Kruskal Wallis followed by Dunn's post hoc test for non-Gaussian distribution).
Abbreviations: ACE inhibitors, angiotensin converting enzyme inhibitors; AR antagonists, angiotensin II receptors antagonists; BMI, body mass index; CAD, coronary artery disease; COPD, chronic obstructive pulmonary disease; CVD, cardiovascular disease; HbA1C, glycated hemoglobin A1C.

TABLE 4
Human primers used to quantify gene expression
using quantitative RT-PCR

| Target | | |
|---|---|---|
| gene | Forward | Reverse |
| ANGPTL2 | CCCCAACACCTTCCACTAAG | AACAGAATCCAGCATCCCG |
| | (SEQ ID N: 27) | (SEQ ID NO: 28) |
| TNFα | CTCTTCTGCCTGCTGCACTT | CTCTCAGCTCCACGCCATTG |
| | (SEQ ID NO: 29) | (SEQ ID NO: 30) |
| IL-8 | CTCTTGGCAGCCTTCCTGAT | TTCTGTGTTGGCGCAGTGTG |
| | (SEQ ID NO: 31) | (SEQ ID NO: 32) |
| IL-6 | GACAGCCACTCACCTCTTCA | CACCAGGCAAGTCTCCTCAT |
| | (SEQ ID NO: 33) | (SEQ ID NO:34) |
| p21 | GGACCTGTCACTGTCTTGTA | CCTCTTGGAGAAGATCAGCCG |
| | (SEQ ID NO: 35) | (SEQ ID NO: 36) |
| GAPDH | AATCCCATCACCATCTTCCA | AAATGAGCCCCAGCCTTC |
| | (SEQ ID NO: 37) | (SEQ ID NO: 38) |

Note:
ANGPTL2, angiopoietin-like 2;
TNF-α, Tumor necrosis factor;
IL-8, interleukin 8;
IL-6, interleukin 6.

### Example 2. Targeting Angptl2 in Severely Dyslipidemic ATX Mice

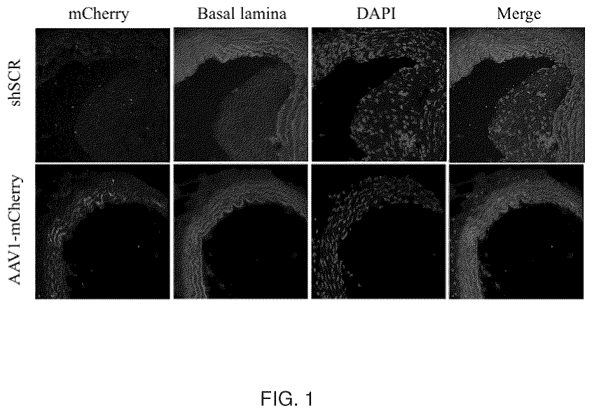

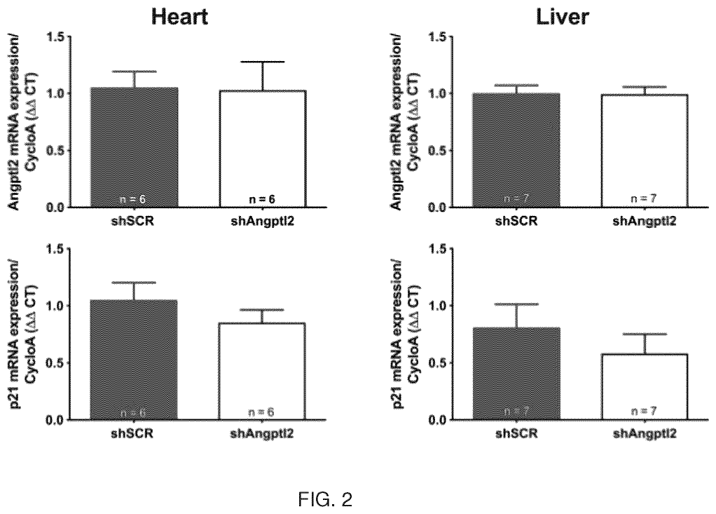

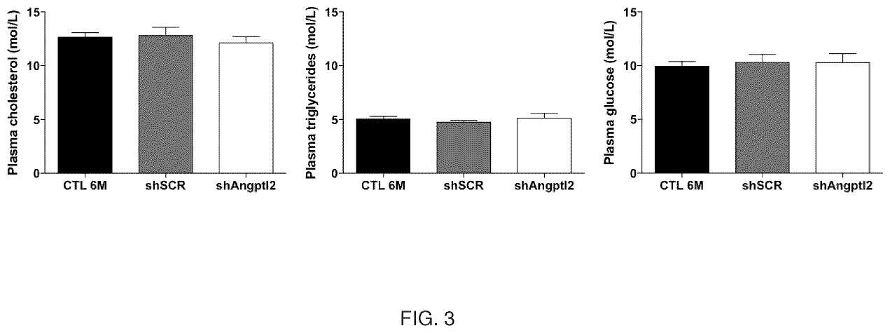

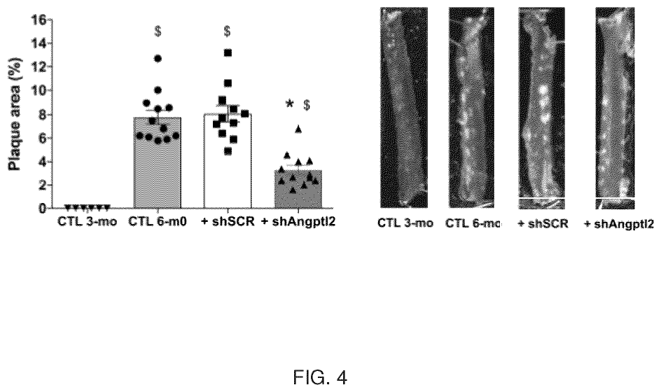

Using AAV1 (1011 viral particles) as vector, shAngptl2 was delivered once (i.v. bolus injection) with preferred vascular tropism (Chen et al., Hum Gene Ther, 16: 235-247, 2005) to 3-month old (-mo) ATX mice. Vascular delivery of the shRNA was confirmed by mCherry staining of the aortic wall with no diffusion to the interstitial space (FIG. 1), while AAV1-shAngptl2 injection did not reduce angptl2 expression in the mouse heart and liver (FIG. 2). The AAV1-shAngptl2 also did not affect lipid and glucose blood levels (FIG. 3). At 6-mo, 3 months post-injection, the atherosclerotic burden in the thoracic aorta was reduced by 58% (FIG. 4; p<0.0001; n=12) compared to un-injected ATX mice and mice injected with an AAV1 containing a scrambled (SCR) mRNA sequence (Table 1). This confirmed that angptl2 is pro-atherogenic, and targeting of angptl2⁺ EC has an anti-atherogenic effect.

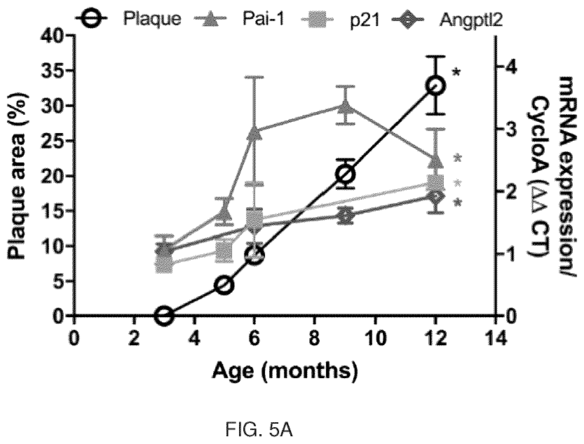

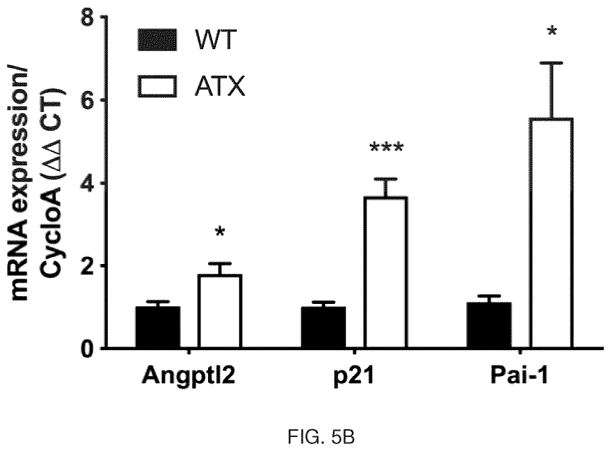

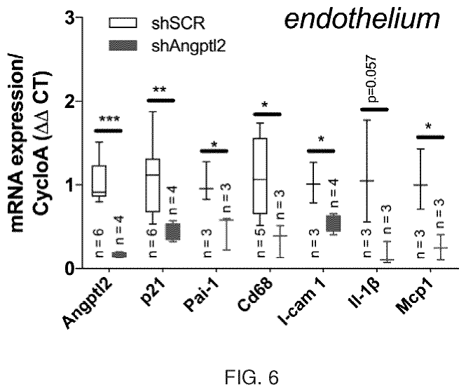

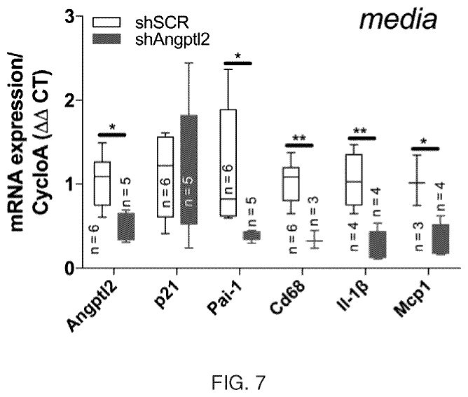

To determine if down-regulation of angptl2 affects senescence, native endothelium p21 and Pai-1 expression was quantified. Expression of angptl2, Pai-1 and p21 paralleled the growing atheroma plaque in ATX mice (FIG. 5A), while all these markers were over-expressed in the endothelium of 6-mo ATX mice compared with WT mice (FIG. 5B). Three months post-AAV1-shAngptl2 injection, angptl2 (p=0.009), Pai-1 (p=0.037) and p21 (p=0.019) expression were significantly decreased in the endothelium (FIG. 6). This finding indicated that targeting angptl2⁺ EC likely reduces SnEC load in vivo. It also suggests that angptl2⁺ EC are senescent, and SnEC favor adhesion of monocytes and their migration in the intima. This is further supported by reduction in the endothelial expression of the adhesion molecule Icam-1 (p=0.05) and the vascular expression of monocyte chemo-attractant protein Mcp1 (FIGS. 6, 7), which likely lowers adhesion of CD68⁺ monocytes (FIG. 6). Taking into account the significance of this pathway in macrophage infiltration and atheroma growth, the aforementioned results demonstrated that lowering angptl2⁺ SnEC burden translates into a lower EC activation. However, p21 expression was not altered in the media of the aorta 3 months post-infection (FIG. 7), suggesting that in this mouse model and at this young age, arterial wall cells and foam cells are not senescent. Angptl2 and CD68 expression, however, decreased (FIG. 7), as did Pai-1, II-1β and Mcp1, suggesting that CD68⁺ macrophages express angptl2 and contribute to the local inflammation. Reduced endothelial CD68 expression is an indication of less monocyte adhesion, reduction of CD68 in the aortic wall indicates reduced angptl2+ macrophage infiltration, and reduced adhesion and infiltration indicates an improved endothelial function 3 months post-AAV1-shAngptl2 therapy.

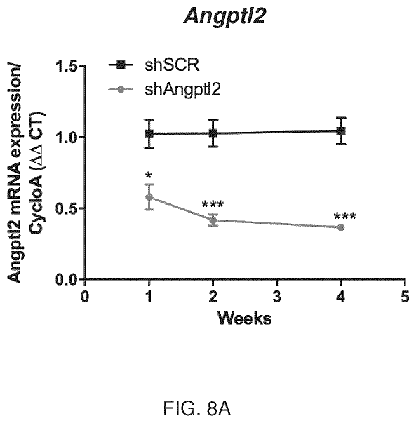

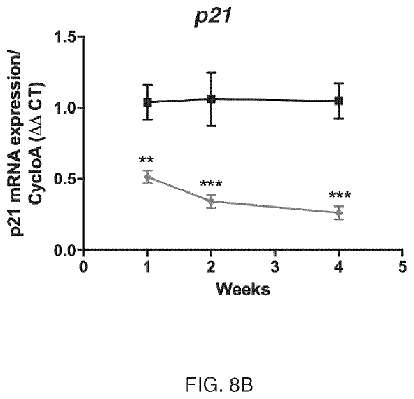

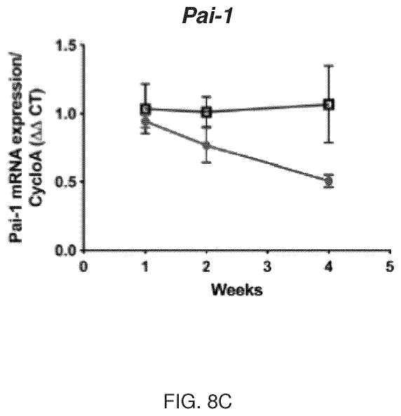

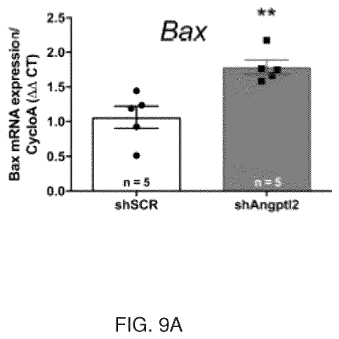

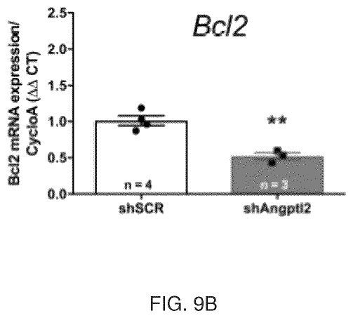

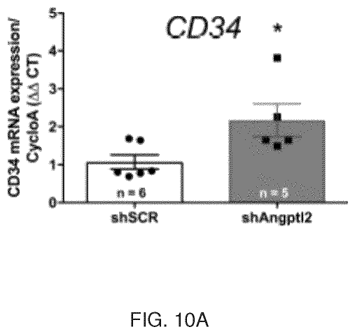

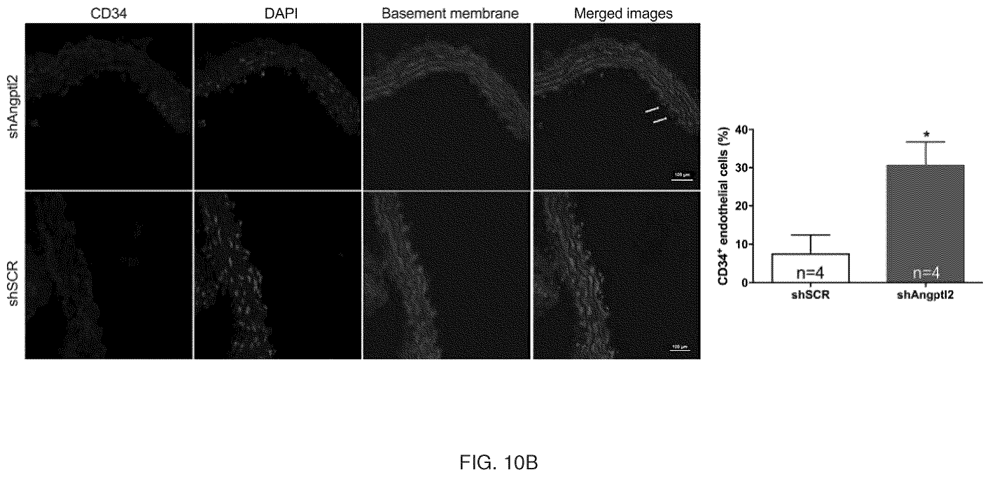

To determine the mechanism of SnEC clearance and endothelial repair associated with the anti-atherogenic effect of the shAngptl2, ATX mice were euthanized 1, 2 and 4 weeks after AAV1-shAngptl2 infection. Endothelial expression of angptl2 (FIG. 8A) and p21 (FIG. 8B) were found to be lowered after one week, and continued to decline over the 4-week period (1-week, n=5/6; 2-week, n=5/7; 4-week, n=4/7), while Pai-1 expression (n=3) tended to decrease after 4 weeks (FIG. 8C). Concomitantly, after one-week, endothelial expression of pro-apoptotic Bax (FIG. 9A) was significantly increased (p=0.0049), while that of the anti-apoptotic Bcl2 (FIG. 9B) was significantly decreased (p=0.0027), indicating that angptl2⁺ SnEC were eliminated following activation of the apoptosis pathway, which is an effective mean of remodelling. Moreover, 2 weeks after the shAngptl2 treatment, CD34 expression tended to increase in the endothelium (from 1.1±0.3 to 1.9±0.3, p=0.082, n=5 and 7, respectively), while it increased significantly (p=0.0342) after 4 weeks (FIG. 10A). This increase in CD34⁺ progenitor cell recruitment was maintained at the end of the study period, i.e., 3 months post-injection (FIG. 10B).

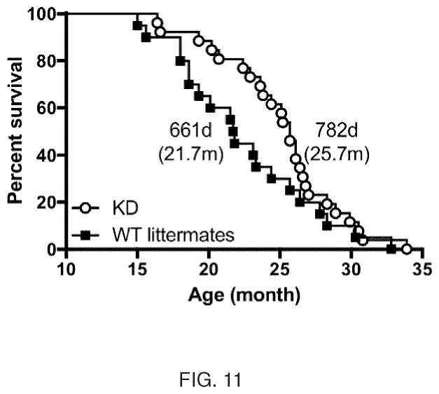

These data evidence that apoptosis of angptl2⁺ SnEC stimulates endothelial repair in part by incorporating circulating EpC. Also, targeting vascular angptl2 appeared to be a safe therapeutic option, as Angptl2-KD mice were healthy, resisted better to inflammatory stress (including in brain vessel, as demonstrated by Yu et al., Am J Physiol Heart Circ Physiol, 308: H386-397, 2015) and had an extended health span (FIG. 11). Hence, taken together, the results indicated that therapeutic down-regulation of vascular angptl2 leads to the clearance of SnEC by apoptosis, stimulates endothelial repair and reduces hepatic steatosis, and also demonstrated that targeting angptl2 could be a selective and safe senolytic strategy.

### Example 3. Accumulation of Arterial SnC Precedes Atherosclerosis in Human

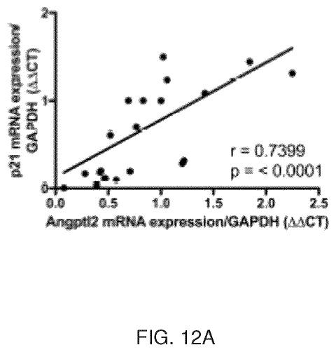

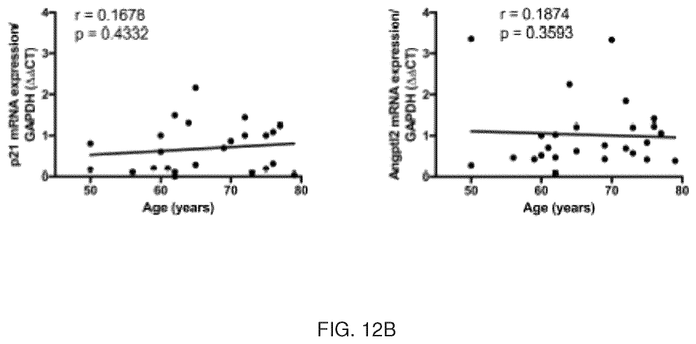

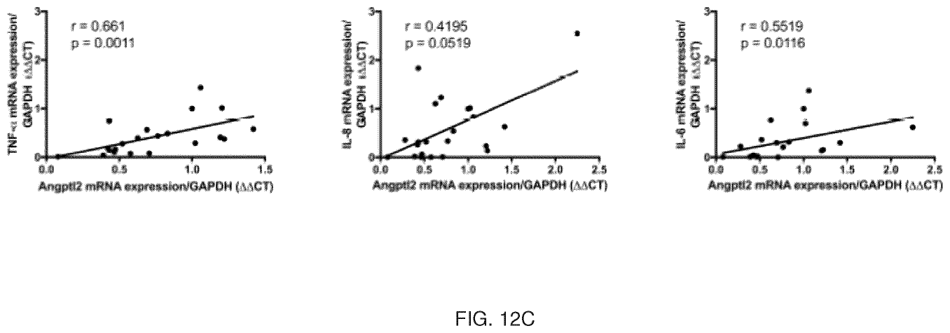

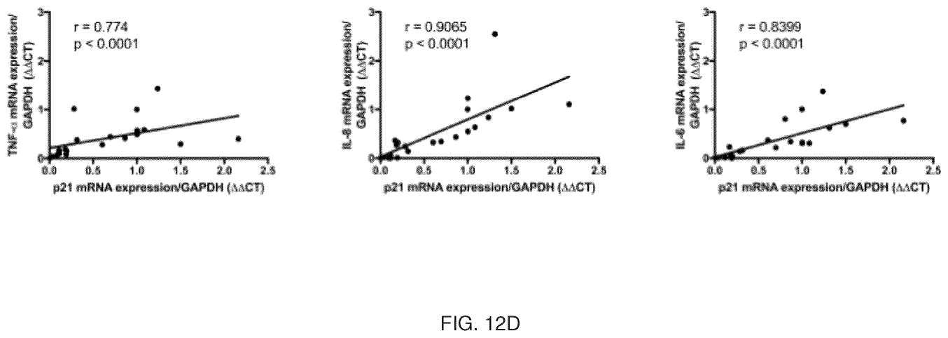

Expression of ANGPTL2 with markers of senescence was measured in functional human arterial segments to assess whether SnC precede plaque growth. Patients (n=26) undergoing CABG surgery with or without valve replacement (Table 3) were recruited. In internal mammary artery segments, the expression of p21 and that of ANGPTL2 were tightly associated (FIG. 12A), while none correlated with the age of the donor (FIG. 12B). Furthermore, ANGPTL2 (FIG. 12C) and p21 (FIG. 12D) levels correlated with pro-inflammatory cytokines of the SASP, i.e., TNFα, IL-8 and IL-6 levels (FIGS. 12C, 12D).

These clinical data validated the strong link between vascular ANGPTL2 and the marker of senescence p21 in yet non-atherosclerotic arteries, independent of age, indicating that accumulation of arterial SnC precedes atherosclerosis.

### Example 4. Treatment of Atherosclerosis in a Subject

One or more methods and/or compositions described herein is used to treat atherosclerosis in a subject (e.g., a human) by inducing cell death (e.g., apoptosis) of angptl2⁺ senescent cells (e.g., angptl2⁺ senescent cardiac cells and/or angptl2⁺ SnEC). Agents (e.g., shRNA) that induce cell death (e.g., apoptosis) of angptl2⁺ senescent cells (e.g., angptl2⁺ senescent cardiac cells and/or angptl2+ SnEC) or a composition containing the same is administered to a subject, such as a human (e.g., a human with atherosclerosis) in order to treat atherosclerosis. Administration of the one or more angptl2⁺ senescent cell targeting agent or a composition containing the same, for instance, can reduce angptl2 expression, reduce senescence-associated secretory phenotype (SASP, such as Pai-1), decrease inflammation, reduce atherosclerotic lesions, reduce atherogenesis, increase endothelial repair, increase cardiac repair, reduce growth of atheroma plaque, reduce atherosclerotic burden (e.g., in thoracic aorta), reduce incidence and/or risk of developing atrial fibrillation, reduce incidence and/or risk of heart failure, reduce incidence and/or risk of developing vascular cognitive impairment and dementia (VCID), and/or increase lifespan of the subject. For instance, a human with atherosclerosis is treated by administering one or more angptl2⁺ senescent cell targeting agent (e.g., an shRNA, such as shAngptl2, or a vector (e.g., an AAV1 or AAV9) containing the same) or a composition containing the same by an appropriate route (e.g., intravenously) in a particular dose (e.g., a dose containing a therapeutically effective amount of the agent), such as from 10 μg/kg to 500 mg/kg (e.g., 10 μg/kg, 50 μg/kg, 100 μg/kg, 150 μg/kg, 200 μg/kg, 250 μg/kg, 300 μg/kg, 350 μg/kg, 400 μg/kg, 450 μg/kg, 500 μg/kg, 1 mg/kg, 10 mg/kg, 50 mg/kg, 100 mg/kg, 150 mg/kg, 200 mg/kg, 250 mg/kg, 300 mg/kg, 350 mg/kg, 400 mg/kg, 450 mg/kg, or 500 mg/kg of the agent) one or more times daily, weekly, every two weeks, every three weeks, or monthly. A single dose or more than one dose of the one or more angptl2⁺ senescent cell targeting agent described herein or a composition containing the same is administered to the subject over a course of days, weeks, months, or years.

The angptl2⁺ senescent cell targeting agent or a composition containing the same is administered to the subject in an amount sufficient to reduce angptl2 expression, reduce SASP (e.g., Pai-1), decrease inflammation, reduce atherosclerotic lesions, reduce atherogenesis, increase endothelial repair, increase cardiac repair, reduce growth of atheroma plaque, reduce atherosclerotic burden (e.g., in thoracic aorta), reduce incidence and/or risk of developing atrial fibrillation, reduce incidence and/or risk of heart failure, reduce incidence and/or risk of developing VCID, and/or increase lifespan of the subject by 10% or more (e.g., 10%, 20%, 30%, 40%, 50%, 60%, 70%, 80%, 90%, 95% or more). The progression of atherosclerosis that is treated with the angptl2⁺ senescent cell targeting agent described herein or a composition containing the same is monitored by any one or more of several established methods. A physician can monitor the subject by direct observation in order to evaluate how the symptoms exhibited by the subject have changed in response to treatment (e.g., by evaluation of atherosclerotic lesions in the subject). Based on such observations, a physician may prescribe higher/lower dosages or more/less frequent dosing of the angptl2⁺ senescent cell targeting agent or a composition containing the same in subsequent rounds of treatment.

### Example 5. Treatment of Hepatic Steatosis in a Subject

One or more methods and/or compositions described herein are useful for treating hepatic steatosis (e.g., NASH) in a subject (e.g., a human) by inducing cell death (e.g., apoptosis) of angptl2+ senescent cells (e.g., angptl2⁺ senescent hepatocyte and/or angptl2⁺ SnEC). Agents (e.g., shRNA) that induce cell death (e.g., apoptosis) of angptl2⁺ senescent cells (e.g., angptl2⁺ senescent hepatocyte and/or angptl2⁺ SnEC) or a composition containing the same is administered to a subject, such as a human (e.g., a human with hepatic steatosis) to treat hepatic steatosis. Administration of the one or more angptl2⁺ senescent cell targeting agent or a composition containing the same, for instance, is useful to reduce angptl2 expression, reduce SASP, decrease inflammation, reduce liver triglyceride level, increase endothelial repair, increase hepatocyte repair, and/or increase lifespan of the subject. For instance, a human with hepatic steatosis is treated by administering one or more angptl2⁺ senescent cell targeting agent (e.g., an shRNA, such as shAngptl2, or a vector (e.g., an AAV8) containing the same) or a composition containing the same by an appropriate route (e.g., intravenously) in a particular dose (e.g., a dose containing a therapeutically effective amount of the agent), such as from 10 μg/kg to 500 mg/kg (e.g., 10 μg/kg, 50 μg/kg, 100 μg/kg, 150 μg/kg, 200 μg/kg, 250 μg/kg, 300 μg/kg, 350 μg/kg, 400 μg/kg, 450 μg/kg, 500 μg/kg, 1 mg/kg, 10 mg/kg, 50 mg/kg, 100 mg/kg, 150 mg/kg, 200 mg/kg, 250 mg/kg, 300 mg/kg, 350 mg/kg, 400 mg/kg, 450 mg/kg, or 500 mg/kg of the agent) one or more times daily, weekly, every two weeks, every three weeks, or monthly. A single dose or more than one dose of the one or more angptl2⁺ senescent cell targeting agent described herein or a composition containing the same is administered to the subject over a course of days, weeks, months, or years.

The angptl2⁺ senescent cell targeting agent or a composition containing the same is administered to the subject in an amount sufficient to reduce angptl2 expression, reduce SASP, decrease inflammation, reduce liver triglyceride level, increase endothelial repair, increase hepatocyte repair, and/or increase lifespan of the subject by 10% or more (e.g., 10%, 20%, 30%, 40%, 50%, 60%, 70%, 80%, 90%, 95% or more). The progression of hepatic steatosis that is treated with the angptl2⁺ senescent cell targeting agent described herein or a composition containing the same is monitored by any one or more of several established methods. A physician typically monitors the subject by direct observation in order to evaluate how the symptoms exhibited by the subject have changed in response to treatment (e.g., by evaluation of liver triglyceride level in the subject). Based on such observations, a physician may prescribe higher/lower dosages or more/less frequent dosing of the angptl2⁺ senescent cell targeting agent or a composition containing the same in subsequent rounds of treatment.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

What is claimed is:

1. A method of treating a disease associated with an angiopoietin like-2 (angptl2) positive (angptl2⁺) senescent cell in a subject in need thereof, the method comprising administering to the subject an agent that induces cell death of the angptl2⁺ senescent cell.

2. The method of claim 1, wherein the cell is a hepatocyte, a cardiac cell, a glial cell, a neuron, a synovial cell, or an endothelial cell (EC).

3. The method of claim 2, wherein the cell is an EC.

4. The method of any one of claims 1-3, wherein the agent is a vector encoding a small hairpin RNA (shRNA).

5. The method of claim 4, wherein the vector is a viral vector.

6. The method of claim 5, wherein the viral vector is an adeno-associated virus (AAV).

7. The method of any one of claims 1-6, wherein the cell is a cardiac cell.

8. The method of any one of claims 1-7, wherein the agent is an AAV serotype 1 (AAV1) or AAV serotype 9 (AAV9) encoding a shRNA.

9. The method of any one of claims 1-8, wherein the disease is a cardiovascular disease.

10. The method of claim 9, wherein the cardiovascular disease is atherosclerosis.

11. The method of claim 10, wherein the method reduces atherosclerotic lesions in the subject.

12. The method of claim 10 or 11, wherein the method reduces atherogenesis in the subject.

13. The method of any one of claims 9-12, wherein the method further comprises administering a second therapeutic agent.

14. The method of claim 13, wherein the second therapeutic agent is an antihypertensive agent or a cholesterol lowering agent.

15. The method of any one of claims 1-6, wherein the cell is a hepatocyte.

16. The method of any one of claims 1-6 and 15, wherein the agent is an AAV serotype 8 (AAV8) encoding a shRNA.

17. The method of any one of claims 1-6, 15 and 16, wherein the disease is a hepatic disease.

18. The method of claim 17, wherein the hepatic disease is hepatic steatosis.

19. The method of claim 18, wherein the hepatic steatosis is non-alcoholic steato-hepatosis (NASH).

20. The method of claim 18 or 19, wherein the method reduces liver triglyceride level in the subject

21. The method of any one of claims 1-6, wherein the cell is a brain cell.

22. The method of any one of claims 1-6 and 21, wherein the disease is a cerebrovascular disease.

23. The method of claim 22, wherein the cerebrovascular disease is vascular cognitive impairment and dementia (VCID).

24. The method of any one of claims 1-6, wherein the disease is an autoimmune disease.

25. The method of claim 24, wherein the autoimmune disease is arthritis or psoriasis.

26. The method of any one of claims 1-25, wherein the cell death is apoptosis.

27. The method of any one of claims 1-26, wherein the method reduces mRNA expression of angptl2.

28. The method of any one of claims 1-27, wherein the method reduces protein expression of angptl2.

29. The method of any one of claims 1-28, wherein the method increases endothelial repair.

30. The method of any one of claims 1-29, wherein the method reduces senescence-associated secretory phenotype (SASP).

31. The method of any one of claims 1-30, wherein the method reduces inflammation.

32. The method of any one of claims 1-31, wherein the method increases lifespan of the subject.

33. The method of any one of claims 1-32, wherein the subject is a human.

34. A pharmaceutical composition comprising an agent that induces cell death of an angptl2+ senescent cell for use in treating a disease associated with angptl2⁺ senescent cells in a subject in need thereof.

35. The pharmaceutical composition for use according to claim 34, wherein the agent is a vector encoding a shRNA.

36. The pharmaceutical composition for use according to claim 35, wherein the vector is a viral vector.

37. The pharmaceutical composition for use according to claim 36, wherein the viral vector is an AAV.

38. The pharmaceutical composition for use according to any one of claims 34-37, wherein the agent is an AAV1 or AAV9 encoding a shRNA.

39. The pharmaceutical composition for use according to any one of claims 34-38, wherein the disease is a cardiovascular disease.

40. The pharmaceutical composition for use according to claim 39, wherein the cardiovascular disease is atherosclerosis.

41. The pharmaceutical composition for use according to claim 40, wherein the pharmaceutical composition reduces atherosclerotic lesions in the subject.

42. The pharmaceutical composition for use according to claim 40 or 41, wherein the pharmaceutical composition reduces atherogenesis in the subject.

43. The pharmaceutical composition for use according to any one of claims 39-42, wherein the pharmaceutical composition further comprises a second therapeutic agent.

44. The pharmaceutical composition for use according to claim 43, wherein the second therapeutic agent is an antihypertensive agent or a cholesterol lowering agent.

45. The pharmaceutical composition for use according to any one of claims 34-37, wherein the agent is an AAV8 encoding a shRNA.

46. The pharmaceutical composition for use according to any one of claims 34-37 and 45, wherein the disease is a hepatic disease.

47. The pharmaceutical composition for use according to claim 46, wherein the hepatic disease is hepatic steatosis.

48. The pharmaceutical composition for use according to claim 47, wherein the hepatic steatosis is NASH.

49. The pharmaceutical composition for use according to claim 47 or 48, wherein the pharmaceutical composition reduces liver triglyceride level in the subject

50. The pharmaceutical composition for use according to any one of claims 34-37, wherein the disease is a cerebrovascular disease.

51. The pharmaceutical composition for use according to claim 50, wherein the cerebrovascular disease is VCID.

52. The pharmaceutical composition for use according to any one of claims 34-37, wherein the disease is an autoimmune disease.

53. The pharmaceutical composition for use according to claim 52, wherein the autoimmune disease is arthritis or psoriasis.

54. The pharmaceutical composition for use according to any one of claims 34-53 further comprising a therapeutically acceptable carrier.

55. The pharmaceutical composition for use according to any one of claims 34-54, wherein the cell death is apoptosis.

56. The pharmaceutical composition for use according to any one of claims 34-55, wherein the pharmaceutical composition reduces mRNA expression of angptl2.

57. The pharmaceutical composition for use according to any one of claims 34-56, wherein the pharmaceutical composition reduces protein expression of angptl2.

58. The pharmaceutical composition for use according to any one of claims 34-57, wherein the pharmaceutical composition increases endothelial repair.

59. The pharmaceutical composition for use according to any one of claims 34-58, wherein the pharmaceutical composition reduces SASP.

60. The pharmaceutical composition for use according to any one of claims 34-59, wherein the pharmaceutical composition reduces inflammation.

61. The pharmaceutical composition for use according to any one of claims 34-60, wherein the pharmaceutical composition increases lifespan of the subject.

62. The pharmaceutical composition for use according to any one of claims 34-61, wherein the subject is a human.
